(19) United States
(12) Patent Application Publication
(10) Pub. No.: US 2011/0165565 A1
(43) Pub. Date: Jul. 7, 2011

(54) COMPOSITIONS AND METHODS FOR POLYNUCLEOTIDE EXTRACTION AND METHYLATION DETECTION

(75) Inventors: Tza-Huei Wang, Timonium, MD (US); Stephen Baylin, Baltimore, MD (US); James Gordon Herman, Lutherville, MD (US); Vasudev Bailey, Baltimore, MD (US); Hariharan Easwaran, Baltimore, MD (US); Hetty Carraway, Baltimore, MD (US); Yi Zhang, Baltimore, MD (US); Brian P. Keeley, Rockford, MI (US)

(73) Assignee: THE JOHNS HOPKINS UNIVERSITY, Baltimore, MD (US)

(21) Appl. No.: 12/811,581

(22) PCT Filed: Jan. 5, 2009
(86) PCT No.: PCT/US09/00039

§ 371 Date: Mar. 25, 2011

Related U.S. Application Data
(60) Provisional application No. 61/009,918, filed on Jan. 3, 2008.
(60) Provisional application No. 61/105,100, filed on Oct. 14, 2008.

(51) Int. Cl. C12Q 1/68; C07H 1/06

(52) U.S. Cl. 435/6.11; 435/6.1; 536/25.41

(57) ABSTRACT

The present invention features methods and compositions for methylation detection, as well as a novel method for polynucleotide extraction and sodium bisulfite treatment.

### CROSS-REFERENCE TO RELATED APPLICATION

This application claims the benefit of the following U.S. Provisional Application Nos. 61/009,918, filed Jan. 3, 2008, and 61/105,100, filed Oct. 14, 2008, the entire contents of which are incorporated herein by reference.

### STATEMENT OF RIGHTS TO INVENTIONS MADE UNDER FEDERALLY SPONSORED RESEARCH

This work was supported by the following grants from the National Science Foundation Grant No: DBI-0552063. The government has certain rights in the invention.

### BACKGROUND OF THE INVENTION

In higher eukaryotes, DNA methylation of cytosines in CpG islands forms an important epigenetic mark that is correlated with gene silencing of tumor suppressor genes. DNA methylation plays an important role in cellular development, differentiation, X-chromosome inactivation, imprinting, suppression of transposable elements, aging and tumor progression. Tumorigenesis results from a series of gain-of-function (oncogenes) and loss-of-function (tumor suppressor) changes, both of which are mediated by genetic and/or epigenetic alterations. The most investigated epigenetic modification in cancer is the heritable transcriptional silencing of tumor suppressor genes resulting from DNA methylation of cytosines at the promoter region. Aberrant DNA hypermethylation is observed at classic tumor-suppressor genes, which are known to be genetically mutated and cause inherited forms of cancer. Tumor cells display a larger number of genes inactivated by promoter hypermethylation than by genetic mutations. Furthermore, these abnormal epigenetic changes appear to be an early event that precedes detection of genetic mutations. Thus, detection of promoter hypermethylation is a valuable tool for early diagnosis of cancer, monitoring tumor behavior, as well as measuring response of tumors to targeted therapy.

The number of tools available to assess DNA methylation demonstrates the extensive interest that has been invested in understanding the role of epigenetics in carcinogenesis. One of the more common techniques used for the detection of methylation is methylation-specific PCR (MSP). The technique relies on sodium bisulfite treatment of DNA which converts unmethylated cytosines to uracils, while leaving methylated cytosines unaffected. The modified sequences are then amplified with specific primers, and the amplified products are identified using gel electrophoresis. Standard MSP approaches are time-consuming, offer only qualitative analysis, and cannot discern relative amounts of methylation. Although real-time PCR-based MSP methods enable quantitative analysis, they are more expensive and require optimization and standardization. In addition, current standard one-step MSP approaches lack the sensitivity for direct screening of challenging samples, such as serum and sputum, where the DNA quantities are minimal, thereby requiring nested amplification PCR steps.

In recent years, several approaches have been used to detect and differentiate methylated regions in normal versus cancer tissues. First generation methods were primarily based on the use of restriction enzymes followed by southern blotting. The usefulness of this approach is limited by the large amount of DNA needed to carry out the analysis. Adequate amounts of DNA are rarely available in samples of serum, sputum, and many other biological samples. Second generation methods are based on either discovering differentially methylated regions in normal versus cancer tissues or analyzing the methylation profile of candidate tumor suppressor genes. Available techniques can be broadly classified into: (a) CpG screening methods, such as MSP, Methylight; (b) genomic screens employing methylation-sensitive restriction enzymes followed by bisulfite conversion, for example in bisulfite sequencing; and (c) gene expression analyses to identify genes that are expressed on reversal of epigenetic modifications by pharmacological agents. Regardless of the category the detection method falls under, the first steps in these second generation methods include DNA extraction followed by sodium bisulfite treatment. The usefulness of these techniques is also limited by the high yield and quality of DNA required, as well as by the efficiency of bisulfite treatment, which is essential for all these techniques. Present methods for DNA extraction, which involve chemical lysis of cells followed by organic solvent extraction and ethanol precipitation, are relatively laborious and time consuming.

Accordingly, improved methods for DNA extraction and detection of methylation are urgently required.

### SUMMARY OF THE INVENTION

As described below, the present invention features methods and compositions for methylation detection, as well as a novel method for polynucleotide extraction and sodium bisulfite treatment.

In one aspect, the invention generally features a method for detection of polynucleotide methylation, the method involves amplifying a polynucleotide containing unmethylated cytosines converted to uracil with a primer pair, where one primer contains a binding moiety having affinity for a binding partner, to obtain an amplicon; capturing the labeled-amplicon with a binding partner fixed to a quantum dot; and inducing fluorescence resonance energy transfer between the quantum dot and the detectable label, thereby detecting polynucleotide methylation.

In another aspect, the invention features a method for quantification of polynucleotide methylation, the method involving amplifying a polynucleotide containing unmethylated cytosines converted to uracil with a primer pair, where one primer contains a binding moiety having affinity for a binding partner, to obtain an amplicon; capturing a labeled-amplicon with a binding partner fixed to a quantum dot; and inducing fluorescence resonance energy transfer between the quantum dot and the detectable moiety, thereby detecting polynucleotide methylation.

In yet another aspect, the invention features a method for detection of polynucleotide methylation, the method involving amplifying a polynucleotide containing unmethylated cytosines converted to uracil with a primer pair, where one primer contains a binding moiety having affinity for a binding partner and the other primer contains a detectable moiety, to obtain an amplicon; capturing a labeled-amplicon with a binding partner fixed to a quantum dot; and inducing fluorescence resonance energy transfer between the quantum dot and the detectable moiety, thereby detecting polynucleotide methylation.

In still another aspect, the invention features a method for detection of polynucleotide methylation, the method involving amplifying a polynucleotide containing unmethylated cytosines converted to uracil with a primer pair, where one primer contains a binding moiety having affinity for a binding partner, and the amplification is carried out using at least one detectably labeled base; capturing a labeled-amplicon with a binding partner fixed to a quantum dot; and inducing fluorescence resonance energy transfer between the quantum dot and the detectable moiety, thereby detecting polynucleotide methylation.

In another aspect, the invention features a method for detection of polynucleotide methylation, amplifying a polynucleotide containing unmethylated cytosines converted to uracil with a primer pair, where one primer contains a binding moiety having affinity for a binding partner; hybridizing a denatured amplicon with a detectably labeled probe to label the amplicon; capturing the labeled-amplicon with a binding partner fixed to a quantum dot; and inducing fluorescence resonance energy transfer between the quantum dot and the detectable moiety, thereby detecting polynucleotide methylation.

In yet another aspect, the invention features a method for detection of DNA methylation, the method involving contacting DNA with a reagent that converts unmethylated cytosines to uracil; amplifying the DNA using forward and reverse primers, where one primer is labeled with a binding moiety and the other is labeled with a fluorophore; capturing a labeled amplicon using a quantum dot containing a binding partner having affinity for the binding moiety; and exciting fluorescence resonance energy transfer between the quantum dot and the fluorophore and detecting fluorophore emission, thereby detecting DNA methylation.

In another aspect, the invention features a method for detection of DNA methylation, the method involving contacting DNA with sodium bisulfite under conditions that provide for the conversion of unmethylated cytosines to uracil; amplifying the DNA using forward and reverse primers, where one primer is labeled with biotin and the other is labeled with a fluorophore; capturing the labeled amplicon using a quantum dot containing streptavidin; and exciting fluorescence resonance energy transfer between the quantum dot donor and the fluorophore acceptor and detecting fluorophore emission, thereby detecting DNA methylation.

In another aspect, the invention features a method for diagnosing or characterizing a disease. The method involves contacting DNA extracted from a biological sample with sodium bisulfite under conditions that provide for the conversion of unmethylated cytosines to uracil;

amplifying the DNA using forward and reverse primers, where one primer is labeled with biotin and the other is labeled with a fluorophore; capturing a labeled amplicon containing biotin and fluorphore using a quantum dot containing streptavidin; exciting fluorescence resonance energy transfer between the quantum dot donor and the fluorophore acceptor and detecting fluorophore emission; and comparing the fluorophore emission with a reference, where detection of an alteration in DNA methylation diagnoses or characterizes a disease.

In another aspect, the invention features a method for diagnosing a neoplasia, the method involving contacting DNA extracted from a biological sample with sodium bisulfite under conditions that provide for the conversion of unmethylated cytosines to uracil; amplifying the DNA using forward and reverse primers, where one primer is labeled with biotin and the other is labeled with a fluorophore; capturing the labeled amplicon using a quantum dot containing streptavidin; and exciting fluorescence resonance energy transfer between the quantum dot donor and the fluorophore acceptor and detecting fluorophore emission, thereby identifying a neoplasia.

In another aspect, the invention features a method for monitoring a disease characterized by an alteration in DNA methylation, the method involving contacting DNA extracted from a biological sample with sodium bisulfite under conditions permissive for the conversion of unmethylated cytosines to uracil; amplifying the DNA using forward and reverse primers, where one primer is labeled with biotin and the other is labeled with a fluorophore; capturing the labeled amplicon using a quantum dot containing streptavidin; exciting fluorescence resonance energy transfer between the quantum dot donor and the fluorophore acceptor and detecting fluorophore emission; and comparing the fluorophore emission with a reference.

In another aspect, the invention features a kit for MS-qFRET detection of DNA methylation, the kit containing reagents for methylation-specific quantum dot fluorescence resonance energy transfer (MS-qFRET) selected from the group consisting of reagents for bisulfite conversion, reagents for PCR amplification, a first primer containing biotin or another binding moiety, a second primer labeled with a detectable moiety, quantum dots (QDs) conjugated to a binding partner for the binding moiety; and instructions. In one embodiment, the instructions are for processing spectral information to determine the level of DNA methylation.

As delineated herein, any of the above aspects of the invention are useful for the detection, quantitation, or characterization of the methylation status of a polynucleotide (e.g., a genomic DNA, a promoter) or for the diagnosis of a disease (e.g., neoplasia, lung cancer, myelodisplastic syndrome). An alteration in methylation status relative to a reference is indicative of the presence of a disease characterized by an alteration in methylation. In one embodiment, the method further involves detecting the methylation status of the polynucleotide in the reaction platform. In one embodiment of the above aspects or any method described herein, a second primer of the pair contains a detectable moiety. In still other embodiments, the amplicon is detectably labeled by hybridization with a detectable probe or by incorporation of a detectably labeled nucleoside. In one embodiment, the binding moiety is a group that mediates ligand binding or a chemically reactive group (e.g., an amine, carboxyl, aldehyde, or sulfhydral group). In other embodiments of the above aspects, the binding moiety and binding partner are biotin/streptavidin, antibody/antigen, or amine-succinimidyl ester. In still other embodiments, fluorophore emission occurs concurrently with quantum dot quenching. In still other embodiments, the polynucleotide is obtained from a biological sample (e.g., any one or more of sputum, stool, blood, blood serum, plasma, cerebrospinal fluid, urine, seminal fluids, ejaculate, and vaginal secretions). In still other embodiments, the method detects an alteration (e.g., an increase or a decrease) in promoter methylation relative to a reference. In one embodiment of any of the above-aspects, the method detects or characterizes a neoplasia in a subject. For example, the method detects or characterizes methylation status of a subject having or having a propensity to develop lung cancer, acute myeloid leukemia, or myelodysplastic syndrome. In still other embodiments, the method characterizes prognosis of a subject having an alteration in methylation. In still other embodiments, the method monitors a tumor or monitors a tumor's responsiveness to therapy. In still other embodiments, the method detects as little as 5, 10, 15 or 20 pg of methylated DNA in the presence of an excess of unmethylated alleles. In still other embodiments, the method detects methylated DNA after as few as 5, 8, 10, or 12 PCR cycles. In still other embodiments, the method provides for quantitative endpoint detection of methylation. In still other embodiments, the method detects methylation status in a polynucleotide isolated from as few as 3-5 cells. In still other embodiments, the method provides for detection of a single quantum dot or a single methylated molecule. In still other embodiments, the method detects DNA methylation in a biological sample obtained from a subject having or at risk of developing lung cancer or myelodysplastic syndrome. In still other embodiments, the method provides for multiplex analyses. In still other embodiments of the above aspects or any aspect of the invention delineated herein, the method further involves amplifying DNA using a second pair of primers, at least one of which contains a fluorophore that is distinguishable from the fluorophore present on the first set of primers. In other embodiments, the method provides for the concurrent analysis of unmethylated and methylated reactions in a single tube. In still other embodiments, a QD donor-acceptor pair is QD525 and BODIPY, QD585 and Alexa594, or QD585 and Cy5. In still other embodiments, methylation is detected using a UV scanner.

In another aspect, the invention features a method for polynucleotide extraction and bisulfite conversion on a single reaction platform, the method involving contacting a sample on a reaction platform with a particle containing a polynucleotide binding agent fixed to a magnetic or magnetizable element under conditions permissive for polynucleotide binding to the particle; isolating the polynucleotide:particle complex on the reaction platform; contacting the polynucleotide:particle complex with a bisulfite reagent under conditions permissive for the conversion of unmethylated cytosines to uracil in the reaction platform; and eluting the bisulfite treated polynucleotide from the particle within the reaction platform.

In another aspect, the invention features a method for polynucleotide extraction and bisulfite conversion in a single reaction vessel, the method involving contacting a sample with a silica particle containing a magnetic or magnetizable element under conditions permissive for polynucleotide binding to the silica particle in a reaction vessel; subjecting the silica particle to a magnetic field to isolate the polynucleotide:silica particle complex; contacting the polynucleotide:silica particle complex with a bisulfite under conditions permissive for the conversion of unmethylated cytosines to uracil; and eluting the bisulfite treated polynucleotide from the silica particle.

In another aspect, the invention features a method for polynucleotide extraction and bisulfite conversion in a single reaction vessel, the method involving contacting a sample with silica superparamagnetic particles (SSP) in a reaction vessel; isolating the SSP:DNA complex in the reaction vessel using a magnetic field; contacting the DNA with bisulfite in the reaction vessel under conditions permissive for the conversion of unmethylated cytosines to uracil; adjusting pH or salt conditions to induce formation of an SSP:DNA complex in the reaction vessel; isolating the SSP:bisulfite converted DNA complex in the reaction vessel using a magnetic field; and eluting the DNA from the SSP.

In one embodiment of the above-aspects or any aspect of the invention delineated herein, the reaction platform is a reaction vessel (e.g., a tube, well, droplet, through-holes, micro or nanofluidic device) or a reaction substrate (e.g., a membrane, filter, fiber, bead, gel matrix, chip, or glass slide). In various embodiments of the above aspects, steps (a) and (d) are carried out at about pH 5-6.5 to permit SSP:DNA binding. In still other embodiments, step (f) is carried out at about pH 8-11.

In another aspect, the invention provides a kit for methylation on beads, the kit containing any one or more of protease K, silica superparamagnetic particles (SSP), a washing buffer, and reagents for sodium bisulfite. In one embodiment, the kit further contains directions for carrying out methylation on beads. In still other embodiments, the method further involves detecting the methylation status of the polynucleotide in the reaction vessel. In still other embodiments, DNA methylation is detected using MS-qFRET or gel electrophoresis. In still other embodiments, the method increases DNA yield from 1000 to 7,000 percent relative to column based extraction, method provides for detection of methylation in DNA extracted from about 10 μL whole blood or in DNA extracted from about 200 μL of serum. In still other embodiments, the method yields about 40 to 70 ng/μL from about 200 μL of serum. In still other embodiments, the elution yield is about 70%, 75%, or 80% of the input DNA. In still other embodiments, the bisulfite conversion efficiency at four hours is about 20% or 25%. In still other embodiments, the sample is a biological sample or laboratory sample. In still other embodiments, the average recovery was at least about 70%, 75%, or 80%. In still other embodiments, the method requires about 4 hours.

The invention generally provides diagnostic methods and compositions for DNA extraction, bisulfite treatment, and methylation detection. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “binding moiety” is meant a portion of a molecule having affinity for another molecule. The affinity may be high affinity or low affinity, so long as it is sufficient to bring the molecules into proximity or to mediate complex formation. Affinity between binding partners may be mediated by virtually any intermolecular forces, such as ionic bonds, hydrogen bonds and Van der Waals forces.

By “bisulfite reaction” or “bisulfite conversion” is meant a reaction for the conversion of a cytosine base in a nucleic acid to an uracil base in the presence of bisulfite ions. Preferably, 5-methyl-cytosine bases are not significantly converted. This is typically accomplished by the bisulfite reaction described by Frommer et al., Proc Natl Acad Sci USA 89 (1992) 1827-31, where cytosine reacts with bisulfite to form a sulfonated cytosine reaction intermediate prone to deamination resulting in a sulfonated uracil, which can be desulfonated to uracil under alkaline conditions. Uracil has the base pairing behavior of thymine, whereas 5-methylcytosine has the base pairing behavior of cytosine. This makes the discrimination of methylated or non-methylated cytosines possible by bisulfite genomic sequencing (Grigg, G., and Clark, S., Bioessays 16 (1994) 431-6; Grigg, G. W., DNA Seq 6 (1996) 189-98), methylation specific PCR (MSP) disclosed in U.S. Pat. No. 5,786,146, or methylation-specific quantum dot fluorescence resonance energy transfer (MS-qFRET). The bisulfite reaction is also described, for example, in Benyajati et al., Nucleic Acids Res 8 (1980) 5649-67 and Olek et al., Nucleic Acids Res 24 (1996) 5064-6 and in U.S. Patent Publication No. 2004/0241704 and 2007/0190530. By “quantum dot” is meant a semiconductor comprising electrons whose movement is constrained in three-dimensions. The quantum dot comprises nanocrystals whose electrical conductivity is altered by an external stimulus. In one embodiment, the nanocrystals comprise elements of periodic groups II-VI, III-V, or IV-VI (e.g., cadmium, zinc, tellurium, selenium and sulfur). In another embodiment, the quantum dot ranges in size from 0.5 to 500 nanometers, 1-100 nanometers, or 2-10 nanometers. The quantum dot's conductivity may be altered, for example, by voltage, photon flux, or any other stimulus known in the art. Upon excitation by a stimulus the quantum dots emit light, for example, at wavelengths from about 470 to 730 nm. In one embodiment, the quantum dot is functionalized with a binding moiety. For example, the quantum dot comprises streptavadin, which facilitates binding with biotin, or an amine, which facilitates succinimidyl ester binding.

In one embodiment, the binding partners may be, for example, complementary nucleic acids, epitopes and antibodies, ligands and proteins, biotin and streptavidin, chemically reactive entities, or metal ions and metal ligands. The binding or reaction between partners can involve the formation of binding pairs from corresponding binding partners attached to two different components, or through the formation of attachments via chemical reactions. As used herein, the term “binding partner” refers collectively to both situations, such that it refers to both a member of a binding pair, as well as either one of two “participants” in an attachment-forming chemical reaction (such as a nucleophile and an electrophile). Examples of chemically reactive pairs that react with one another either directly or by activation in the presence of another reagent, such as a catalyst include, for example, amine/aldehyde, amine/succinimidyl esters, amine/isothiocyanates, amine/terafluorophenyl esters, amine/sulfonyl chlorides, thiol/maleimides, thiol iodoacetamides, aldehyde/hydrazines, aldehyde/hydroxylamines, hydroxyl/carboxyl (with a carbodiimide coupling agent), amine/carboxyl (with a carbodiimide coupling agent). Other reactive groups are well known in the chemical arts.

By “silica superparamagnetic particle (SSP)” is meant a silica micro- or nanoparticle comprising a metal core that may be magnetized. In one embodiment, the SSP comprises a superparamagnetic iron oxide core. In another embodiment, the SSP is about 10, 20, 30, 50, 75, 100, 200, 250, 300, 400, or 500 nm in diameter. Methods for making SSPs are known in the art and are described, for example, by Zhiqing et al., Anal. Chem., 2008, 80 (4), pp 1228-1234; Stjerndahl M et al., Langmuir. 2008 Apr. 1; 24(7):3532-6; and Lou et al., J Mater Sci Mater Med. 2008 January; 19(1):217-23. SSPs are also commercially available (e.g., Qiagen).

By “magnetic” as used herein to refer to SSP, includes materials which are paramagnetic or superparamagnetic materials. The term “magnetic”, as used herein, also encompasses temporarily magnetic materials, such as ferrimagnetic materials. Except where indicated otherwise below, the SSPs used in this invention preferably comprise a superparamagnetic core coated with siliceous oxide, having a hydrous siliceous oxide adsorptive surface (i.e. a surface characterized by the presence of silanol groups).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the level of a marker (e.g., methylation) as detected by standard art known methods such as those described herein. As used herein, an alteration includes a 5% or 10% change, a 15%, 20% or 25% change, a 40% change, a 50% or even greater change in marker level.

By “biologic sample” is meant any tissue, cell, fluid, or other material obtained or derived from an organism.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard of comparison. For example, the methylation level present at a promoter in a neoplasia may be compared to the level of methylation present at that promoter in a corresponding normal tissue.

By “diagnostic” is meant any method that identifies the presence of a pathologic condition or characterizes the nature of a pathologic condition (e.g., a neoplasia). Diagnostic methods differ in their sensitivity and specificity. While a particular diagnostic method may not provide a definitive diagnosis of a condition, it suffices if the method provides a positive indication that aids in diagnosis.

By “increased quantity of methylation” is meant a detectable positive change in the level, frequency, or amount of methylation. Such an increase may be by 5%, 10%, 20%, 30%, or by as much as 40%, 50%, 60%, or even by as much as 75%, 80%, 90%, or 100%.

“Detect” refers to identifying the presence, absence or amount of the agent to be detected.

By “detectable label” is meant a composition that when linked to a molecule of interest renders the latter detectable, via spectroscopic, photochemical, biochemical, immunochemical, or chemical means. For example, useful labels include fluorophores, radioactive isotopes, magnetic beads, metallic beads, colloidal particles, fluorescent dyes, electron-dense reagents, enzymes (for example, as commonly used in an ELISA), biotin, digoxigenin, or haptens. Detectable labels include, but are not limited to Cy5, BODIPY, Alexa594, BOBO-3, POPO-1, BOBO-1, YOYO-1, TOTO-1, JOJO-1, POPO-3, LOLO-1, YOYO-3, and TOTO-3.

Methods of the invention provide for the detection of methylation specific PCR products. The PCR products described herein are rendered detectable by any means known in the art. In one embodiment, PCR is carried out using a primer comprising a detectable label. In another embodiment, PCR is carried out and the resulting amplicon is rendered detectable by hybridization with a detectably labeled probe (termed a hanger probe). In this approach, PCR is carried out with one primer having a binding moiety and one unlabeled primer. The resulting PCR product, which comprises a binding moiety, is then denatured and allowed to hybridize with short fluorescent labeled oligos. In yet another embodiment, the resulting amplicon is rendered detectable by the inclusion of detectably labeled nucleotides in the PCR reaction. The use of fluorescence-labeled nucleotides for PCR according to the invention allows the process to be carried out without the purchase of HPLC purified labeled oligos. If desired, multiple (1, 2, 3, 4, 5, 7, 8, 9, 10) fluorophores are incorporated into one amplified product, thereby eliminating the need for relatively expensive fluorophore labeling of primers. dCTP Cy5 (Cy5 is a commercially obtainable fluorescent dye) can be obtained by Amersham Biotech.

By “disease” is meant any condition or disorder that damages or interferes with the normal function of a cell, tissue, or organ. Examples of diseases include bacterial invasion or colonization of a host cell.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated polynucleotide” is meant a nucleic acid molecule (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “marker” is meant any protein or polynucleotide having an alteration in methylation, expression level, or biological activity that is associated with a disease or disorder.

By “methylation profile” is meant the methylation level at two or more promoters.

By “sensitivity” is meant the percentage of subjects with a particular disease that are correctly detected as having the disease.

By “neoplasia” is meant any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “periodic” is meant at regular intervals. Periodic patient monitoring includes, for example, a schedule of tests that are administered daily, bi-weekly, bi-monthly, monthly, bi-annually, or annually.

By “promoter” is meant a nucleic acid sequence sufficient to direct transcription. In general, a promoter includes, at least, 50, 75, 100, 125, 150, 175, 200, 250, 300, 400, 500, 750, 1000, 1500, or 2000 nucleotides upstream of a given coding sequence

As used herein, “obtaining” as in “obtaining an agent” includes synthesizing, purchasing, or otherwise acquiring the agent.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligonucleotide, more preferably an oligo-deoxyribonucleotide. The primer must be sufficiently long to prime the synthesis of extension products in the presence of the inducing agent. The lengths of the primers will depend on many factors, including temperature, source of primer and the use of the method.

As used herein, the term “purified” or “to purify” means a process or the result of any process which removes some contaminants from the component of interest, such as a DNA extension product. The percent of a purified component is thereby increased in the sample.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reference” is meant a standard or control condition.

By “specificity” is meant the percentage of subjects without a particular disease who test negative.

### BRIEF DESCRIPTION OF THE DRAWINGS

FIG. 1 is a schematic diagram that describes the principle of methylation-specific quantum dot fluorescence resonance energy transfer (MS-qFRET) for detection of DNA methylation. In step 1, extracted genomic DNA is subject to sodium bisulfite conversion, wherein unmethylated cytosines are converted to uracil while methylated cytosines remain unaffected. In step 2, DNA is amplified using PCR wherein the forward and reverse primers are labeled with a biotin (black dot) and a fluorophore (red dot) In step 3, the resulting labeled-PCR product is captured by streptavidin functionalized QDs through streptavidin-biotin affinity. Finally, in step 4, upon suitably exciting the QD, the nanoassembly formed allows for FRET to occur between the QD donor and the fluorophore acceptor. Consequently, the labeled-PCR products are detected by emissions of fluorophores accompanied by quenching of QDs to reveal the status of DNA methylation.

FIGS. 2A-2D are four panels showing the high analytical sensitivity facilitated by inherent low-background noise. FIG. 2A is a graph showing that methylation for p16 can be detected as early as 8 cycles (dark gray curve) as demonstrated by the acceptor (Cy5) emission at 670 nm. Signal from the standard 35 cycle control (gray curve) reflects a much stronger acceptor emission accompanied by stronger QD quenching. Curve from the water control (light gray) shows no acceptor emission.

FIG. 2B shows a corresponding MSP gel readout, which indicated no visible band at 8 cycles for methylated p16 product, but a clear band was observed after the standard 35 cycles. FIG. 2C provides two panels showing results using confocal spectroscopy to observe differences in the positive control (IVD only) and negative control (NL only) through 2,000 ms single-particle traces. Top Panel: In positive control, each Cy5 peak seen (red) is the fluorescence burst associated with labeled-MSP products that is linked to a single QD passing through the focal detection volume of a confocal spectroscopy setup. Bottom panel: The negative control has very low background noise. FIG. 2D shows results obtained using confocal spectropy. IVD was serially diluted in NL DNA (150 ng) and subject to MS-qFRET with 40 cycles of amplification. Confocal spectroscopy was used to analyze fluorescent bursts for the acceptor (Cy5), and was plotted for the entire time duration (3 separate runs of 100 s) for 1/10, 1/100, 1/1,000 and 1/10,000 and 0 methylated/unmethylated p16 alleles (IVD/NL). This indicated the successful detection of methylation with as little as 15 pg of methylated DNA (˜5 genomic equivalents) in 150 ng excess unmethylated DNA.

FIGS. 3A-3E show that MS-qFRET can be used to quantitate methylation in experiments using different ratios of unmethylated and methylated DNA. Increasing percent p16 methylation levels were accompanied by an increase in acceptor (Cy5) emission at 670 nm and corresponding donor (QD605) quenching at 605 nm. FIG. 3B is a graph showing q-scores plotted for the varying levels of p16 methylation. A linear fit was observed with r²=0.999. FIG. 3C is a graph showing MS-qFRET quantitation used to estimate p16 methylation reversal in DNA from RKO cells treated with DAC for different time points. q-scores indicated a drop in the level of methylation post-treatment. FIG. 3D is a graph showing a quantification of methylation reversal at p15 using MS-qFRET in 6 myelodysplastic syndrome (MDS) patients during their first cycle of epigenetic therapy. Changes in levels of methylation were effectively captured to show varying cellular responses to 5-azacytidine and MS-275. FIG. 3E is a graph showing methylation in patients 1-6.

FIGS. 4A-4B show results of multiplex reactions and direct detection. In FIG. 4A the methylated p16 control (M) shows acceptor emission peak of Alexa594 at 620 nm (red trace). The unmethylated p16 control (U) shows Cy5 emission peak at 670 nm (green trace). Multiplexed unmethylated and methylated reaction (U+M) show emission peaks at both 620 nm and 670 nm. No acceptor emission was observed for the water control. In FIG. 4B direct visualization shows FRET (observed through QD quenching) for all genes only in IVD, but not in NL or water control.

FIGS. 5A-5C show the detection of methylation in human sputum samples. FIG. 5A shows a representative gel from sputum DNA. Results, which were obtained using conventional MSP for ASC/TMS1 for 8 patients, indicate the presence of only unmethylated products. In contrast, an electrophoresis gel from nested MSP products detects methylation in Patient 3, 7 and 8. FIG. 5B shows representative fluorescence spectra from 2 patients with differing methylation status. Significant acceptor (Cy5) emission at 670 nm was observed for patients with methylated ASC/TMS1 promoter. FIG. 5C show normalized FRET efficiencies (En) for 20 patients, conducted in a blinded fashion, which indicated that Patient 3, 7 and 8 have methylation for ASC/TMS1. An arbitrary En cut-off of 0.1 is used to determine positive methylation. All patients show unmethylated ASC/TMS1 as well.

FIG. 6 provides a schematic illustration of MOB methylation detection. Step 1: Samples (serum, sputum, tumors etc.) are lysed with protease K at 70° C. SSPs and buffers are added, binding the DNA to the SSPs. Step 2: The tube is placed in a magnetic field to hold the solid phase and bound DNA to the side of the tube as the supernatant is removed by pipette. Wash buffers and bisulfite reagents are added and then removed within a magnetic field in similar fashion. Step 3: PCR buffer is used to elute the bisulfite treated DNA from SSPs and also serves as the reagent for the following MSP reaction. Step 4: After MSP, the samples are analyzed using MS-qFRET or gel electrophoresis.

FIGS. 7A-7D show that MOB yield comparisons to conventional and commercial DNA extraction and bisulfite treatment. FIG. 7A is a graph showing the DNA yield of extraction using MOB compared to conventional phenol ethanol extraction. Increases in DNA yield ranges from a 3,500 to 7,000-percentile increase. FIG. 7B is a graph that provides a comparison of average DNA extraction recovery between MOB and commercial column based extraction (ng/μl). FIG. 7C is a table showing a comparison of yields from MOB, commercial, and conventional bisulfite treatment. FIG. 7D shows a MSP gel electrophoresis analysis of volunteered DNA using MOB.

FIG. 8 is a table showing bisulfite conversion efficiency (%). In particular, the table shows results of a real time MSP analysis of bisulfite treatment of DNA using MOB. Three samples were separated into equal aliquots and analyzed for the bisulfite efficiency of variable incubation durations. The p16 gene was analyzed. Bisulfite conversion efficiency at four hours incubation is comparable to the conventional 16 hour treatment (without the use of kits and columns). Since Ct values for all hours are almost identical, this demonstrates that only four hours is required for the MOB technique for efficient bisulfite conversion.

FIGS. 9A-9C show serum and sputum DNA methylation detection and comparison to tumor status. FIG. 9A shows DNA yield of extraction (ng/μL) from serum using MOB compared to conventional extraction. FIG. 9B shows the DNA yield of extraction (ng/μL) from sputum using MOB compared to conventional extraction. Samples obtained voluntarily from lung cancer patients. FIG. 9C shows the methylation status of p16 from serum, sputum, and tumor is compared to methylation status analyzed through nested MSP.

FIGS. 10A-10C are schematic diagrams illustrating the principle of Ms-qFRET. FIG. 10A shows the tree representative target NAs. FIG. 10B shows that labeled DNA is locally concentrated as streptavidin functionalized AD605 added to the DNA. FIG. 10C shows that QD605 captures biotinylated DNA and forms an assembly where Cy5 dyes are FRET acceptors with QD donor and fluoresce upon QD excitation at 488 nm. The Forster radius RO was calculated to be 64.7 Å.

FIG. 11 is a schematic diagram illustrating the incorporation of dCTP Cy5 during PCR with methylation specific primers and their detection with FRET.

FIGS. 12A-12D compare the detection of p15, RassF1A, CDH13, ASC/TMS1, and p16 genes using single Cy5 labeled amplicons (amplicons generated using a Cy5 labeled primer) and amplicons labeled using dCTP Cy5 relative to control. FIG. 12A shows fluorescence intensity of single Cy5 labeled PCR products, dCTP Cy5 labeled products, and control. FIG. 12B is a table showing percent enhancement of intensity. FIG. 12C is a graph showing FRET efficiency for PCR products of various lengths for p15, RassF1A, CDH13, ASC/TMS1, and p16 genes using a single Cy5 label. FIG. 12D shows FRET efficiency for PCR products labeled using dCTP Cy5. The use of dCTP Cy5 provided for a significant improvement in detection regardless of the length of the PCR product.

FIG. 13 shows the advantage provided by MS-qFRET in differentiating signal from noise related to primer dimers.

FIG. 14 is a graph showing that when the optical detection limit of a fluorescence reader is higher than the intrinsic fluorescence background of QD-FRET, the overall assay sensitivity is limited by the optical sensitivity of instrument. The performance of QD-FRET assay is improved by using a fluorescence detector with high optical sensitivity.

FIGS. 15A and 15B show a comparison of optical detectors. FIG. 15A shows that when detecting QD-FRET DNA mixtures with a spectrophotometer (Nanodrop 3300), a detection limit of 1 nM is achieved. This is limited to the optical sensitivity of the instrument. FIG. 15B shows that further diluted samples are still unambiguously detected with an APD-based fluorescence spectroscope. This result indicates that due to the low intrinsic background of QD-FRET, the performance of the MS-QFRET assay is best when a fluorescent detector of high optical sensitivity is used.

### DETAILED DESCRIPTION OF THE INVENTION

The invention features compositions and methods that are useful for polynucleotide extraction and bisulfite conversion and methylation detection.

The invention relating to DNA preparation is based, at least in part, on the discovery of a single-tube method for polynucleotide extraction and bisulfite conversion, termed “methylation-on-beads (MOB),” which is a rapid and highly efficient method for DNA extraction, bisulfite treatment and detection of DNA methylation using silica superparamagnetic particles (SSP), where all steps are implemented without centrifugation or air drying that provides superior yields relative to conventional methods for DNA extraction and bisulfite conversion. SSP serve as solid substrate for DNA binding throughout the multiple stages of each process. Specifically, SSP are first used to capture genomic DNA from raw tissue samples, processed tissue samples or cultured cells. Sodium bisulfite treatment is then carried out in the presence of SSP without tube transfers. Finally, the bisulfite treated DNA is analyzed to determine the methylation status. DNA extraction yield was found to be 35-55 times the yield from conventional extraction. 90% of the input DNA was recovered after bisulfite treatment. In addition, MOB total process time was completed in less than 6 hours when compared to 3 days for conventional methods. MOB was extended to analyzing DNA methylation in serum, sputum and tumor samples from patients with Stage I and Stage II lung cancer. Less than a third of the initial sample was utilized for analysis. Methylation in serum and sputum in 11/12 patients corresponded with analysis in tumor samples. Hence, MOB allows for convenient, efficient and contamination-resistant methylation detection in a single tube or other reaction platform.

The invention also features compositions and methods that are useful for the qualitative and quantitative detection of methylated DNA, as well as for the detection of low-abundance methylated DNA. This aspect of the invention is based, at least in part, on the discovery that MS-qFRET (Methylation-specific quantum dot FRET) provides an ultrasensitive, reliable nanotechnology assay for detection and quantification of DNA methylation. In this technique, quantum dots are used to capture methylation-specific PCR (MSP) amplicons and to determine the methylation status via fluorescence resonance energy transfer (FRET). Desirably, MS-qFRET has low intrinsic background noise, high resolution and high sensitivity. MS-qFRET detects as little as 15 pg of methylated DNA in the presence of a 10.000-fold excess of unmethylated alleles, enables reduced use of PCR (8 cycles), and allows for multiplexed analyses. To illustrate these capabilities, patient sputum samples containing very low concentrations of methylated DNA were directly tested for promoter methylation at ASC/TMS1, and bypassed the need for nested MSP. Furthermore, the ability of MS-qFRET to quantify methylation changes with high resolution was demonstrated in cells treated with 5-aza-2′-deoxycytidine and clinical samples from patients with myelodysplastic syndrome (MDS). The favorable attributes of MS-qFRET allow for broad applications in both clinical and research settings, and permit convenient and simple methylation detection. The direct application of MS-qFRET on clinical samples offers great promise for its translational use in early detection of cancer diagnosis, prognostic assessment of tumor behavior, as well as monitoring response to therapeutic agents.

### Extraction Methods of the Invention

Many solid malignancies may not cause symptoms until the tumors have metastasized. Therefore, a biomarker-based detection of cancer can be more useful for early detection, thereby resulting in an improved clinical outcome. In particular, DNA methylation analysis of tumor-free DNA in the bloodstream, epithelial tumor cells shed in a lumen and in sputum offer promising ways for early detection of cancer. Existing methods for DNA methylation detection lack of sensitivity resulting in false negatives. DNA methylation detection presents great difficulties in sputum and serum due to the small quantity of DNA found in these biological samples. Methylation detection is currently conducted through ethanol precipitation extraction, followed by conventional or commercial bisulfite treatment and nested MSP. DNA yields from both serum and sputum are incredibly variable due to the imprecise methods of collecting sputum and can lead to inadequate amounts of DNA for testing. As a result, DNA methylation is dependent upon the limitations of traditional techniques and requires meticulous implementation of commercial protocols. Compounding the small amounts of DNA found in many biological samples, current methods are susceptible to DNA loss associated with the methodology. For example, DNA loss occurs during multiple tube transfers, column chromatography; pipetting, unsuccessful binding to a column, or incomplete elution from the column. Such losses in combination with the small amount of DNA present in the sample can compromise detection.

Currently, DNA isolation and bisulfite treatment are independent processes, which require transferring sample between reaction tubes. Due to multiple sample transfers and column based purification, the yield after bisulfite treatment is not satisfactory. As reported in more detail below, the present invention addresses the loss of DNA during processing by combining DNA isolation, bisulfite treatment and downstream PCR based analysis into one single procedure.

DNA binds to silica surfaces in chaotropic salt solutions, such as those containing iodide or perchlorate salt. Taking advantage of the chaotropic salt induced DNA adsorption, one can easily isolate DNA other cellular components. In a typical protocol, cell lysates are mixed with a silica substrate in a chaotropic solution, which promotes binding of DNA to the silica surface. Other macromolecules such as proteins and lipids remain unbound in the solution and are then removed by separating the solid phase from the solution. Additional washing steps with alcohol are required to ensure the DNA purity for further analysis. The bound DNA is then eluted in low ionic strength buffer. Typically, the solid substrate is a matrix or gel that is fixed in a column. It can also be in the form of particles and the separation is realized by centrifugation. The present invention substitutes silica superparamagnetic particles (SSP) for the solid matrix. The use of SSP simplifies sample handling and obviates the need for centrifugation. The superparamagnetic solid substrate is separated from the solution with an external magnetic field.

The magnetic manipulation offers great potential for system integration and automation and less potential for contamination. In addition, because SSP is used as the solid substrate for DNA manipulation in every stage of processing, the extraction and purification can be carried out in a single tube. The single tube format minimizes sample transfer and retains DNA while simultaneously decreasing contamination and improving yields significantly. This technique was first successfully employed using as little as 10 μL whole blood. This technique has also been used for the efficient and complete bisulfite conversion by measuring and comparing Ct values using SSP with results obtained using conventional bisulfite conversion. Finally, this technique was successfully used to extract DNA from serum and sputum samples obtained from patients with Stage I and II lung cancer. The total yield and efficiency in recovery after bisulfite conversion were significantly higher than both commercial kits as well as the conventional treatment methods.

### Diagnostic Assays

The present invention provides methods and compositions for DNA extraction and DNA methylation detection. Such methods are useful in a number of diagnostic assays. In particular, such methods and compositions are useful for the identification or characterization of epigenetic changes in a biological sample associated with neoplasia, such as. e.g. lung cancer or acute myeloid leukemia) and other diseases characterized by alterations in methylation, such as MDS (myelodysplastic syndrome). In one embodiment, a biological sample (e.g., sputum, serum, cells, tissue) is characterized by extracting DNA from the biological sample using the MOB approach described herein or any other extraction method known to the skilled artisan, and quantifying or determining the methylation level of DNA isolated from the neoplasia. In one embodiment, methylation levels are determined using MS-qFRET to detect CpG methylation in genomic DNA. Methods for identifying CpG islands are described, for example, in U.S. Patent Publication No. 2006/0240460 and 2006/0019267. MS-qFRET uses sodium bisulfate to convert unmethylated cytosine to uracil. A comparison of sodium bisulfate treated and untreated DNA provides for the detection of methylated cytosines.

While the examples provided below describe methods of detecting methylation levels using MS-qFRET, the skilled artisan appreciates that the invention is not limited to such methods. Methylation levels are quantifiable by any standard method, such methods include, but are not limited to quantitative methylation specific PCR (QMSP), real-time PCR, Southern blot, bisulfite genomic DNA sequencing, restriction enzyme-PCR, MSP (methylation-specific PCR), methylation-sensitive single nucleotide primer extension (MS-SNuPE) (see, for example, Kuppuswamy et al., Proc. Natl. Acad. Sci. USA, 88, 1143-1147, 1991), DNA microarray based on fluorescence or isotope labeling (see, for example, Adorján Nucleic Acids Res., 30: e21 and Hou Clin. Biochem., 36:197-202, 2003), mass spectroscopy, methyl accepting capacity assays, and methylation specific antibody binding. See also U.S. Pat. Nos. 5,786,146, 6,017,704, 6,300,756, and 6,265,171.

The primers used in the invention for amplification of the CpG-containing nucleic acid in the specimen, after bisulfite modification, specifically distinguish between untreated or unmodified DNA, methylated, and non-methylated DNA. Methylation specific primers for the non-methylated DNA preferably have a T in the 3′ CG pair to distinguish it from the C retained in methylated DNA, and the complement is designed for the antisense primer.

The primers of the invention embrace oligonucleotides of sufficient length and appropriate sequence so as to provide specific initiation of polymerization on a significant number of nucleic acids in the polymorphic locus. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting methylated nucleic acid.

In one approach, methylation specific primers amplify a desired genomic target using the polymerase chain reaction (PCR).

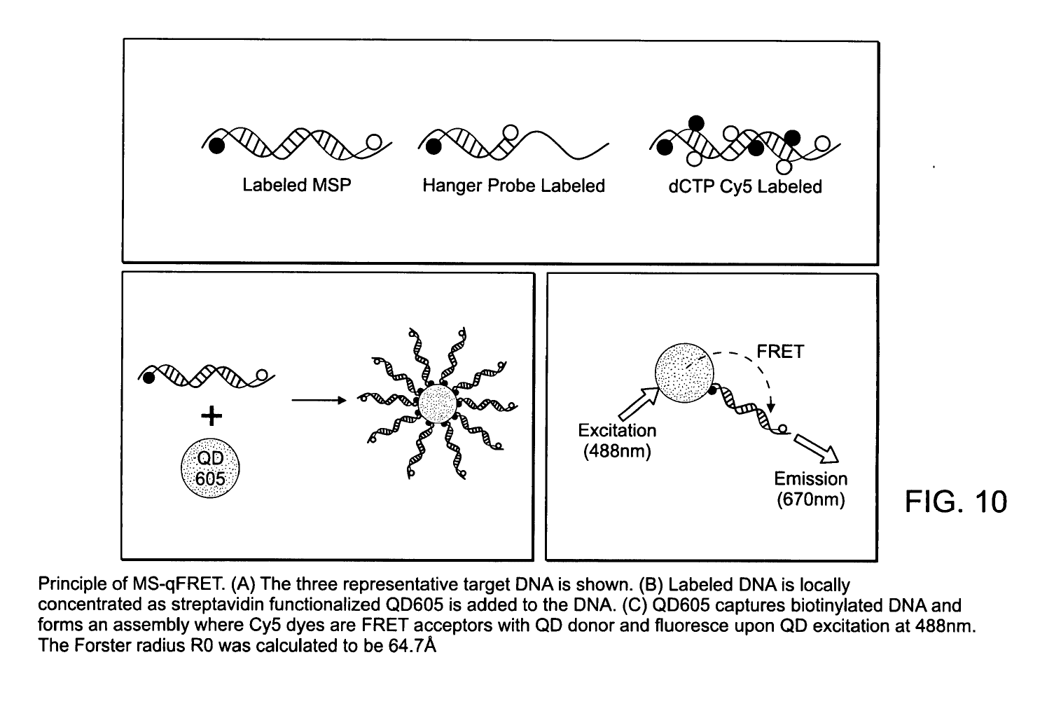

The invention provides improved methods and compositions for the detection of DNA methylation. Such methods are useful not only as research tools, but also as diagnostics for the characterization of clinical samples. In general, the methods of the invention involve, subjecting extracted genomic DNA to sodium bisulfite conversion, where unmethylated cytosines are converted to uracil while methylated cytosines remain unaffected. DNA is then amplified using methylation specific PCR. In one approach, forward and reverse primers are labeled with a binding moiety and a detectable moiety. The resulting labeled-PCR product (i.e. amplicon) is captured by a quantum dot functionalized to include a binding partner having affinity for the binding moiety on the amplicon. In another approach, one of the primer pair is labeled with a binding moiety and the other primer is unmodified. The resulting PCR product is rendered detectable by inclusion of detectable nucleotides (e.g., dCTP CY5) during the amplification reaction (FIG. 11). Methods for carrying out the incorporation of detectable nucleotides during PCR are known in the art and described, for example, in U.S. Pat. No. 7,153,671. Alternatively, or in addition, the PCR product is rendered detectable by hybridization with a detectable probe, termed a hanger probe.

In one embodiment, a hanger probe is a short (i.e., 16 to 30 base pair) fluorescently labeled oligonucleotide at least a portion of which is complementary to the target amplicon. In one embodiment, the MSP reaction primers employ only one primer that is labeled with a binding moiety. The use of hanger probes allows for an extra level of specificity in detection and eliminates the need to check for primer dimers, thereby allowing this nanoassay to be further adapted for high throughput quantitative screening. Another approach involves the use of dCTP-labeled with Cy5 directly in the MSP reaction. This helps to streamline the process, and also enhances detection sensitivity due to the presence of multiple acceptors for a single DNA. These alternative methods demonstrate the versatility of the nanoassay in adapting to additional sensitivity and specificity requirements.

Regardless of which approach is adopted, the detectable amplicon, which comprises a binding moiety, may be used in MS-qFRET. Upon suitably exciting the QD, the nanoassembly formed between the QD and the amplion allows for FRET to occur between the QD donor and the fluorophore acceptor. Consequently, the labeled-PCR products are detected by emissions of fluorophores accompanied by quenching of QDs to reveal the status of DNA methylation.

In one embodiment, the moiety that has affinity for a binding partner is biotin and the binding partner is streptavidin. In one approach, a quantum dot is functionalized for binding. Functional moieties include, but are not limited to, components that contain chemically reactive groups such as amines, carboxyl, aldehyde, sulfhydral groups or combinations of such chemically reactive groups; and to components that contain ligand binding or other binding groups such as biotin/streptavidin, antibody/antigen, metal chelating or coordination structures, amine-succinimidyl ester binding, or combinations of those binding groups. In another embodiment, the detectable label is a fluorophore.

The resulting labeled-PCR product is then captured by its binding partner (e.g., streptavidin), which is present on functionalized quantum dots.

The amplified product is then detected using methods of the invention or using standard methods known in the art. In one embodiment, a PCR product (i.e., amplicon) or real-time PCR product is detected by MS-qFRET. Methods for DNA methylation detection are described, for example, in U.S. Patent Publication No. 2006/0183115. The quantum dot provides for detection of the labeled PCR product when the QD is excited by a stimulus. In one embodiment, excitation of the nanoassembly (e.g., QD-PCR product complex) allows FRET to occur between the QD donor and the fluorophore acceptor. The labeled-PCR products are then detected by emissions of fluorophores accompanied by quenching of QDs, thereby detecting the presence or absence of DNA methylation.

DNA or other polynucleotides extracted using the MOB approach are useful in a variety of applications. Methylation-specific PCR products generated from the MOB extracted DNA may be detected using MS-qFRET or any other detection method known in the art. In another embodiment, an amplicon is detected by a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a methylation specific PCR product.

### Types of Biological Samples

Methylation can be measured in different types of biologic samples. In one embodiment, the biologic sample is a biologic fluid sample. Biological fluid samples include sputum, blood, blood serum, plasma, cerebrospinal fluid, urine, stool, seminal fluids, ejaculate, vaginal secretions, or any other biological fluid useful in the methods of the invention. In another embodiment, the biologic sample is a tissue sample that includes cells of a tissue, organ, or tumor obtained, for example, from a biopsy.

The present invention provides methods of diagnosing disease and/or disorders or symptoms characterized by alterations in methylation. In one embodiment the invention provides methods for selecting a treatment regiment for a subject suffering from or susceptible to a disease or disorder or symptom thereof characterized by alterations in methylation. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein (e.g., a compound that modulates methylation), or a composition described herein to produce such effect. Identifying a subject in need of such treatment can be in the judgment of a subject or a health care professional and can be subjective (e.g. opinion) or objective (e.g. measurable by a test or diagnostic method). As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The diagnostic or therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The diagnostic described herein may be used for the diagnosis of any disorders in which alterations in methylation may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof characterized by alterations in methylation, in which the subject has been administered a therapeutic amount of a compound herein sufficient to treat the disease or symptoms thereof. The level of Marker (e.g., methylation) determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Kits

The invention provides kits for the diagnosis or monitoring of a disease characterized by an alteration in methylation. In one embodiment, it provides for the detection of hypermethylation associated with a neoplasia (e.g., lung cancer, myelodysplastic syndrome). In one embodiment, the kit detects an alteration in the level of a Marker (e.g., promoter methylation) relative to a reference level of methylation (e.g. promoter methylation present in a biological sample obtained from a healthy control subject). In related embodiments, the kit includes reagents for monitoring the methylation level of a promoter in a biological sample derived from a subject. In other embodiments, the kit comprises a sterile container which contains a primer, probe, sodium bisulfite, SSP, and/or detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids.

In one embodiment, the kit provides reagents to carry out methylation-specific quantum dot fluorescence resonance energy transfer (MS-qFRET). Such reagents include, but are not limited to, chemicals containing bisulfite for DNA treatment, reagents for PCR amplification, a first primer comprising biotin or another binding moiety, and a second primer labeled with a detectable moiety (e.g., fluorophore), quantum dots (QDs) conjugated to a binding partner for the binding moiety (e.g. streptavidin). If desired, the kit further includes instructions for processing spectral information to determine the level of DNA methylation.

In another embodiment, the invention provides reagents for carrying out methylation on beads, the kit comprising an effective amount of silica superparamagnetic particles (SSP). If desired, the kit further comprises one or more washing buffers and reagents for bisulfite treatment.

In other embodiments, the instructions will generally include information about the use of the primers or probes described herein and their use in detecting methylation or in detecting, diagnosing or monitoring a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a disease characterized by an alteration in methylation (e.g., a neoplasia, such as lung cancer or myelodysplastic syndrome) can be monitored using the methods and compositions of the invention. Such monitoring may be useful, for example, in assessing the efficacy of a particular drug in a patient. Therapeutics that alter the methylation of a promoter are taken as particularly useful in the invention.

### Particles for DNA Purification

Silica based systems have been developed for use in the purification of DNA from other materials (e.g., biological samples, experimental samples). Such systems include those which employ controlled pore glass, filters embedded with silica particles, silica gel particles, resins comprising silica in the form of diatomaceous earth, glass fibers or mixtures of the above. One of skill in the art will appreciate that although the examples provided herein specifically describe the use of silica, the invention is not so limited. In fact, any solid phase agent that binds a polynucleotide, such as genomic DNA, may be used in the methods of the invention, so long as the agent reversibly binds polynucleotides when placed in contact with polynucleotides in the presence of chaotropic agents. Such agents include, but are not limited to, glass surfaces, silica gel, diatomic earths, and organo silane particles. The term“chaotropic agent” as used herein refers to salts of particular ions which, when present in a sufficiently high concentration in an aqueous solution, cause proteins present therein to unfold and nucleic acids to lose secondary structure. It is thought that chaotropic ions have these effects because they disrupt hydrogen-bonding networks that exist in liquid water and thereby make denatured proteins and nucleic acids thermodynamically more stable than their correctly folded or structured counterparts. Chaotropic ions include guanidinium, iodide, perchlorate, and trichloroacetate. Chaotropic agents include guanidine hydrochloride, guanidine thiocyanate (which is sometimes referred to as guanidine isothiocyanate), sodium iodide, sodium perchlorate, and sodium trichloroacetate.

The silica-based solid phases are designed to remain bound to the nucleic acid molecules while the solid phase is exposed to an external force, such as centrifugation or vacuum filtration to separate the matrix and bound nucleic acid material from other materials. The nucleic acid molecules are then eluted from the solid phase by exposing the solid phase to an elution solution, such as water or an elution buffer. Magnetically responsive solid phases, such as paramagnetic or superparamagnetic particles, offer an advantage not offered by other solid phases. Such particles could be separated from a solution by turning on and off a magnetic force field, by moving a container on to and off of a magnetic separator, or by moving a magnetic separator on to and off of a container. Such activities would be readily adaptable to automation. Magnetically responsive particles have been developed for use in the isolation of nucleic acid molecules by the direct reversible adsorption of nucleic acids to the particles. See, e.g., silica gel-based porous particles designed to reversibly bind directly to DNA, such as MagneSil Paramagnetic Particles (Promega), or Biome Paramagnetic Beads (Polysciences, Warrington, Pa., U.S.A.). See also Smith et al., U.S. Pat. No. 6,027,945. Magnetically responsive glass beads of a controlled pore size have also been developed for the isolation of nucleic acids. See, e.g. Magnetic Porous Glass (MPG) particles from CPG, Inc. (Lincoln Park, N.J., U.S.A.); or porous magnetic glass particles described in U.S. Pat. Nos. 4,395,271, Beall et al.; 4,233,169, Beall et al.; or 4,297,337, Mansfield et al.

Nevertheless, the methods of the invention are readily adaptable to use with any method of separating a solid phase agent, such as silica, bound to a polynucleotide. In particular, the methods of the invention are useful not only in a single-tube format, but are readily adaptable to use in any reaction vessel or on any reaction substrate known in the art. Useful substrate materials include membranes, composed of paper, nylon or other materials, filters, fibers, beads, gel matrices, chips, glass slides, and other solid supports. Reaction vessels include, for example, tubes, wells, droplets, through-holes, and micro or nanofluidic devices.

For methods of adsorption and desorption of nucleic acids to silica magnetic particles in general, some of which methods are suitable for use in the present invention, see international patent application number PCT/US98/01149 for METHODS OF ISOLATING BIOLOGICAL TARGET MATERIALS USING SILICA MAGNETIC PARTICLES, published as WO 98/31840, incorporated by reference herein. Adsorption of the DNA extension products to the silica magnetic particles used in the present invention preferably takes place in the presence of an adsorption solution.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### MS-qFRET Provides for PCR Product Detection by Fluorophore Emission

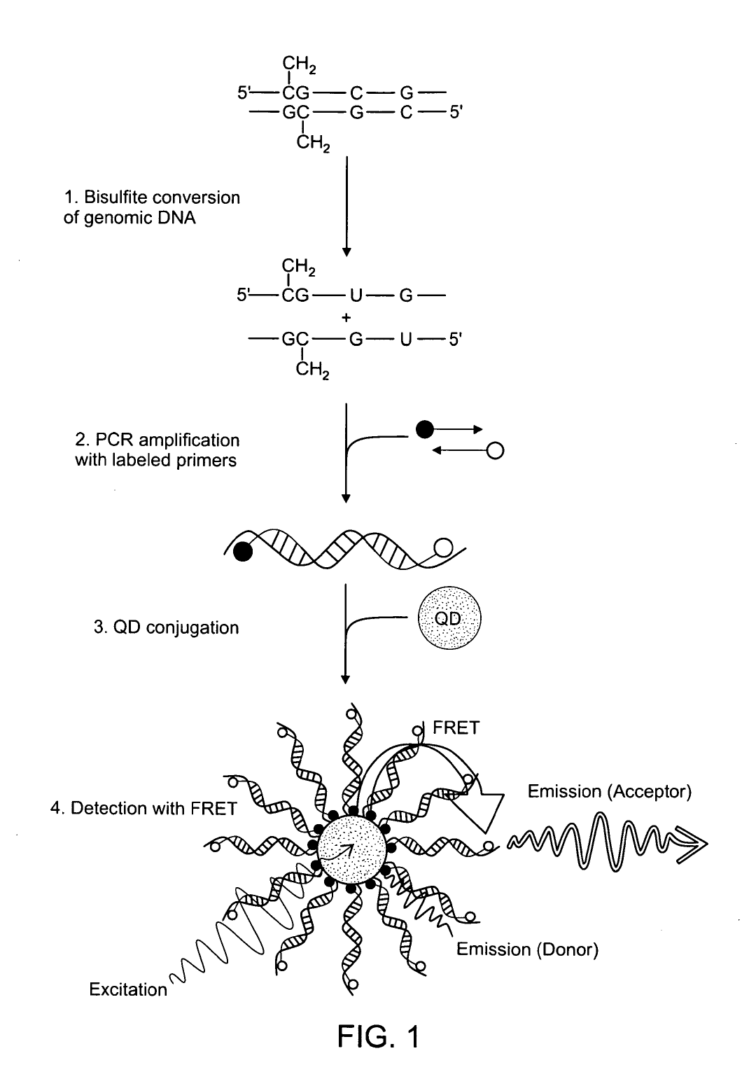

In methylation-specific quantum dot fluorescence resonance energy transfer (MS-qFRET), the bisulfite-treated DNA is amplified through PCR, wherein the forward primer is biotinylated and the reverse primer is labeled with an organic fluorophore (FIG. 1). Next, streptavidin-conjugated quantum dots (QDs) are introduced to capture the labeled PCR products via streptavidin-biotin binding, bringing the QDs (serving as donors) and fluorophores (serving as acceptors) in close proximity allowing FRET to occur. Finally, PCR products are detected by emissions of fluorophores accompanied by quenching of QDs. Spectral information is processed to determine the level of DNA methylation.

### Example 2

### MS-qFRET Detected PCR Products at 8 Cycles of Amplification

To examine the background noise level of MS-qFRET, control experiments were conducted using in-vitro methylated DNA (IVD) and unmethylated DNA (Normal lymphocytes, NL) with methylation-specific primers for the p16 promoter (Table 1).

TABLE 1

| Gene | | |
|---|---|---|
| | Unmethylated Forward | Unmethylated Reverse |
| p15 | 5′-GGTTGGTTTTTTATTTTGTTAGAGTGAGGT-3′ | 5′-AACCACTCTAACCACAAAATACAAACACA-3′ |
| | (SEQ ID NO: 1) | (SEQ ID NO: 2) |
| p16 | 5′-GGTTGGTTTTTTATTTTGTTAGAGTGAGGT-3′ | 5′-AACCACTCTAACCACAAAATACAAACACA-3′ |
| | (SEQ ID NO: 1) | (SEQ ID NO: 2) |
| TMS1 | 5′-GAAGGTGGGGAGTTTAGGTTTTGTTTT-3′ | 5′AAATTCTCCAACACATCCAAAATAACAT-3′ |
| | (SEQ ID NO: 3) | (SEQ ID NO: 4) |
| | Methylated Forward | Methylated Reverse |
| p15 | 5′-GGTTTTTTATTTTGTTAGAGCGAGGC-3′ | 5′-TAACCGCAAAATACGAACGCG-3′ |
| | (SEQ ID NO: 5) | (SEQ ID NO: 6) |
| p16 | 5′-TTATTAGAGGGTGGGGCGGATCGC-3′ | 5′-TAACCGCAAAATACGAACGCG-3′ |
| | (SEQ ID NO: 7) | (SEQ ID NO: 6) |
| TMS1 | 5′-GCGGGGAGTTTAGGTTTCGTTTC-3′ | 5′-CCAACGCATCCAAAATAACGTCG-3′ |
| | (SEQ ID NO: 8) | (SEQ ID NO: 9) |
| | Nested | |
| TMS1-Flank | 5′-GGGAGTTGGGAGATTAGAGT-3′ | |
| up | (SEQ ID NO: 10) | |
| TMS1-Flank | 5′-CAACAACTTCAACTTAAACTTCTTAAACTC-3′ | |
| down | (SEQ ID NO: 11) | |

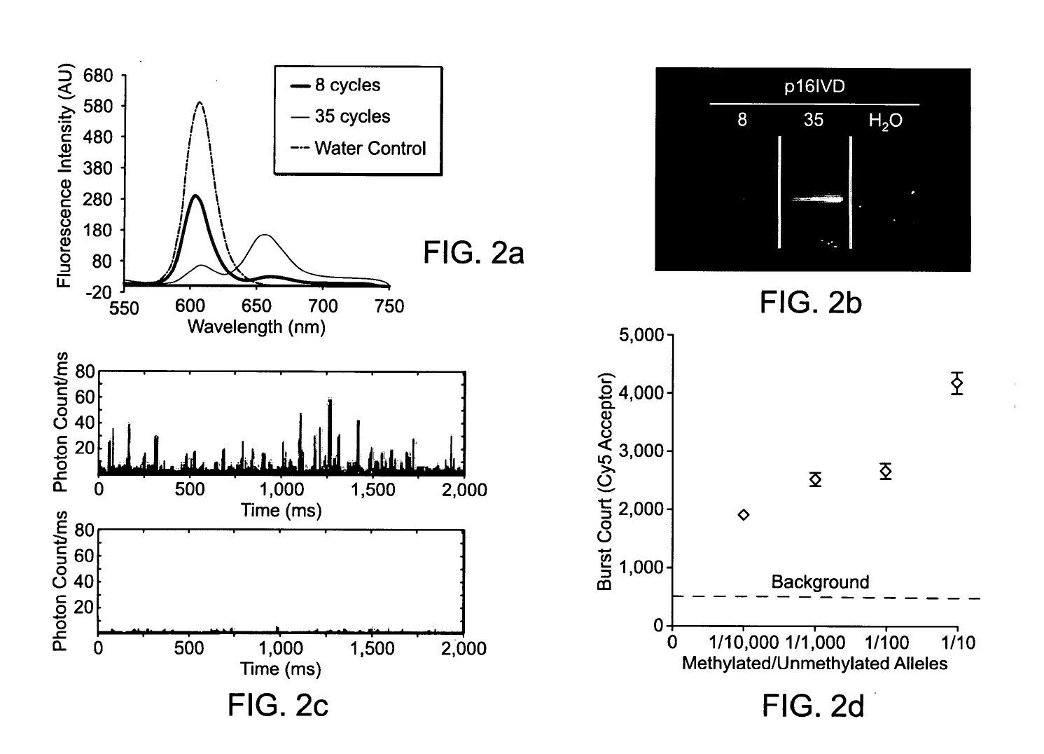

Fluorescence responses were measured using a fluorospectrometer. First, MS-qFRET was noted to detect PCR products as early as 8 cycles of amplification. Starting quantities of DNA typical for MSP (50-150 ng) are described by Herman et al., (1996) Proc Natl Acad Sci USA 93, 9821-6 (FIG. 2a). In contrast, conventional gel or real-time based MSP methods generally require amplification of >20 cycles in order to detect the presence of amplicons (Eads et al. (2000) MethyLight: a high-throughput assay to measure DNA methylation. Nucleic Acids Res 28, E32, Fackler et al. (2004) Cancer Res 64, 4442-52). As seen in FIG. 2A, the low background noise of the water control permits the detection of amplicons at such early cycles. Analysis showed strong FRET signals for a wide range of amplicon sizes that ranged from 68 bp to 151 bp (Table 1). Since most amplicon sizes lie within this range, MS-qFRET can be easily applicable in analyzing various genes. In addition, the Förster distance calculated verifies the feasibility of various acceptor-donor pairs.

| | | |
|---|---|---|
| Donor/Max Emission | Acceptor/Max. Excitation | Rₒ |

| | | | |
|---|---|---|---|
| QD605/605 nm | Cy5/652 | nm | 69.4 Å |
| QD585/585 nm | Alexa594/594 | nm | 54.3 Å |
| QD585/585 nm | Cy5/652 | nm | 59.2 Å |

Förster distance calculations. The FRET pairs used include combinations of a QD donor (QD585 or QD605) and an acceptor (Alexa594 or Cy5). The Förster distance (Rₒ) is calculated using the Förster formalism

$${R}_{0} = {( \frac{9000 ( ln 10 ) {k}_{p}^{2} {Q}_{D}}{{N}_{A} 128 {π}^{5} {n}_{D}^{4}} I )}^{\frac{1}{6}} ,$$

where kₚ^{z} is the orientation factor (⅔ for randomly oriented dipoles), Q_{D} is the quantum yield of QD585 (˜0.2) and QD605 (˜0.6) (Invitrogen Corporation); NA is the Avogadro's number, n_{D} is the refractive index of the medium (1.4 for biomolecules in aqueous solution); I is the normalized overlap integral and is calculated using I=∫₀^{∞}PL_{D-corr}ε_{A}(λ)λ⁴dλ where PL_{D-corr} is the normalized donor emission spectra and ε_{A}(λ) is the acceptor absorption spectra and is expressed as an extinction coefficient. For the 2 organic fluorophores we used, ε_{A}(594 nm)=80,400 cm⁻¹ M⁻¹ for Alexa594 and ε_{A}(647 nm)=250,000 cm⁻¹ M⁻¹ for Cy5.

While most clinical applications can use the conventional fluorospectrometer, the analytical sensitivity of detection can be best defined by a confocal spectroscope, sensitive at the level of single-molecule fluorescence (Wang et al., (2005) J Am Chem Soc 127). Notably, the background level was minimal in the presence of only NL, while strong FRET signals were clearly observed in the presence of IVD, when amplified with methylation-specific primers (FIGS. 2C and D). Confocal spectroscope measurements detected FRET signal using as little as 15 pg (˜5 genomic equivalents) of methylated DNA (IVD) in an excess of 150 ng of unmethylated DNA (NL) (FIG. 2D). It is possible that the true sensitivity could be at a single-molecule level (digital level), given that numerous fluorescent burst counts were clearly observed as compared to the NL controls. The above results demonstrate the ability of MS-qFRET to clearly detect methylation with high sensitivity due to its high signal to noise ratio.

### Example 3

### Quantification of Methylation

The capability of PCR detection at the early log-linear stage makes quantifying

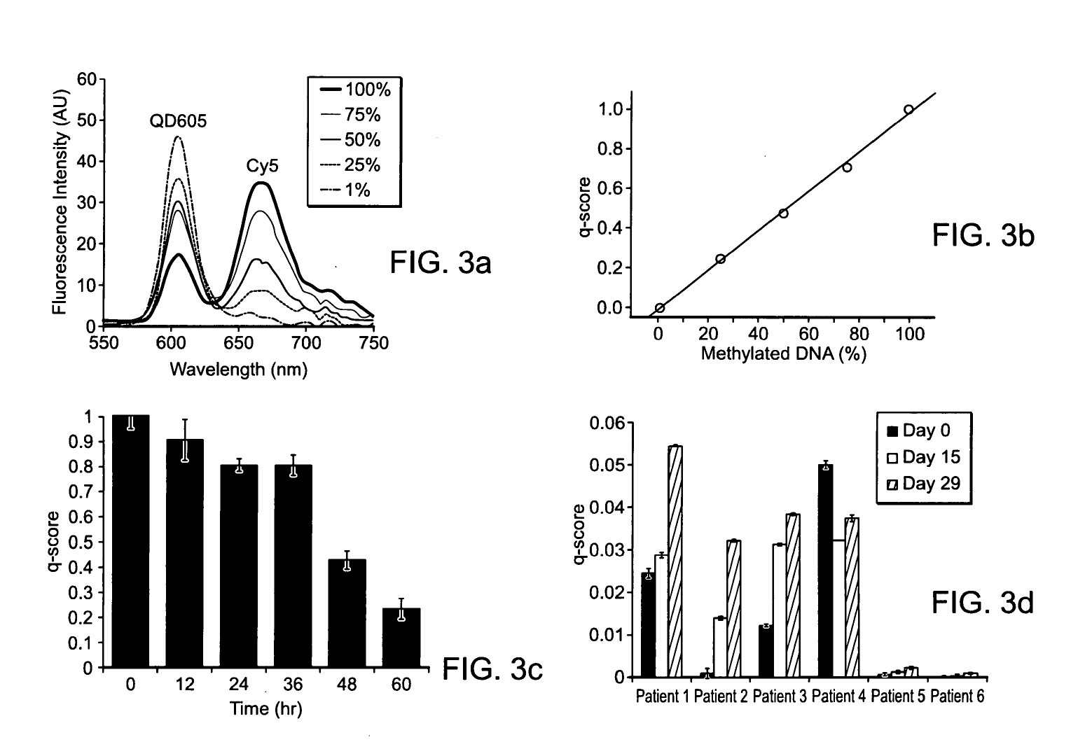

DNA methylation possible. To examine the quantitative accuracy of MS-qFRET, IVD and NL were mixed in different ratios, and analyzed at the p16 promoter with methylation-specific primers. As shown in FIG. 3a, with an increasing amount of input methylated DNA in the mixture (with a fixed total DNA concentration), there is a corresponding increase in the intensity of the acceptor (Cy5) emission, and donor (QD605) quenching. FIG. 3b shows a linear correlation between the normalized FRET efficiency, herein referred to as the q-score (see Methods), and the input methylation level. By including a methylated or unmethylated dilution series in every assay with a known total input DNA, a standard curve can be created to quantify methylation of unknown samples from the q-score. Hence, MS-qFRET can be used as a quantitative technique for methylation analysis.

### Example 4

### Monitoring Methylation Changes after Drug Treatment in Cell Lines and Samples from Patients with Myelodysplastic Syndrome (MDS)

The quantitative ability of MS-qFRET was further tested in cell lines and in patient samples as a function of response to a DNA demethylating agent. Reversal of methylation in the colorectal cancer cell line, RKO, was quantified at specific time points after treatment with 5-aza-2′-deoxycytidine (DAC). FIG. 3C shows a 10 to 20 percent decrease in methylation at p16 within 24 to 36 hours with maximal reversal seen at 60 hours post-treatment. Since DNA replication is necessary for incorporation of DAC into DNA, reversal of methylation may be best observed only after inhibition of DNA methyltransferases due to cell cycling. The low amount of methylation reversal in the initial 24 to 36 hours could be attributed to the 22 hour doubling time for RKO cells. These results are consistent with methylation reversal studies using Ms-SNUPE (Bender et al. (1999) Mol Cell Biol 19, 6690-8).

To demonstrate the quantitative ability of MS-qFRET directly in a clinical setting, reversal of methylation at the p15 promoter (Quesnel et al. (1998) Blood 91, 2985-90; Christiansen et al., (2003) Leukemia 17, 1813-9; Shimamoto et al., (2005) Leuk Res 29, 653-9; Herman et al. (1997) Cancer Res 57, 837-41; Daskalakis et al. (2002) Blood 100, 2957-64) was analyzed on bone marrow aspirate samples from patients with MDS who received epigenetic therapy as part of an Institutional Review Board approved clinical trial. Patients were treated with combination therapy using both Vidaza (5-azacytidine) and Entinostat (MS-275), a histone deacetylase inhibitor. Bone marrow aspirate samples were obtained pre-treatment, day 14 and day 29 of the first cycle of combination therapy. Gel electrophoresis is a common means to monitor the response to such therapy (Christiansen et al., (2003) Leukemia 17, 1813-9, 29, 30). However, after many cycles of PCR amplification, the data does not remain quantifiable and resolving methylation becomes challenging. MS-qFRET allows for computing such differences in methylation by measuring methylation at the early stage of amplification. As shown for 6 patients (FIG. 3D), MS-qFRET is used for detecting and tracking methylation changes for each patient in a quantitative manner, with the Day 0 sample (pre-treatment) being the “control” for the following sample time points (Day 15 and Day 29) for each patient. Although there are no consistent trends in reversal of methylation seen in these patients, similar trends were observed in 16 cycle analysis of MS-qFRET as compared to 40 cycles of real-time PCR (FIG. 3E). These data highlight that individual patients have unique responses to epigenetic agents and that these subtle changes are easily quantifiable using MS-qFRET.

### Example 5

### Detection Through Multiplex Reactions

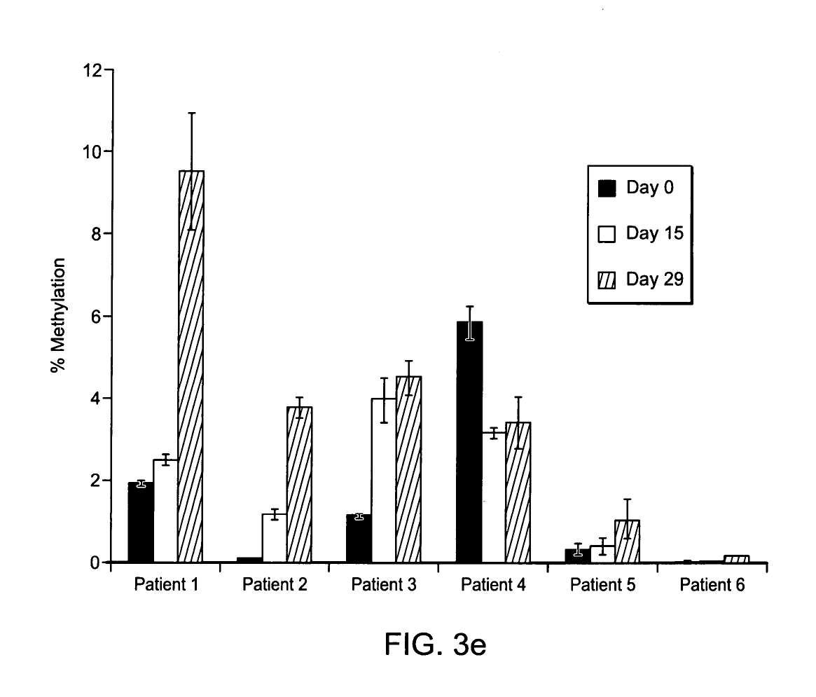

In conventional methylation-specific PCR (MSP) methods (Herman et al., (1996) Proc Natl Acad Sci USA 93, 9821-6; Eads et al. (2000) Nucleic Acids Res 28, E32), each methylated and unmethylated reaction is performed in separate reaction tubes. Using MS-qFRET, simultaneous analysis of both unmethylated and methylated reactions in a single tube was achieved by uniquely labeling unmethylated and methylated p16 primers with Cy5 and Alexa594, respectively. QD585 serves as a common donor to Cy5 and Alexa594 (SI Table 1). As shown in FIG. 4A, the QD585 emission peak was solely observed for the water control, but was quenched in the presence of MSP products. Emission peaks of Cy5 and Alexa594 were observed for unmethylated and methylated DNA respectively. Upon analyzing a mixture of both targets, Cy5 and Alexa594 peaks were simultaneously detected, confirming the presence of both unmethylated and methylated alleles.

### Example 6

### Detection Through Direct Visualization

Recognizing that often a subjective, and yet rapid, determination of methylation is necessary, MS-qFRET can be adapted such that methylation can be detected through simple fluorescent visualization. In both IVD and NL, a qualitative, visual analysis of methylation of p15, p16 and TMS1 promoters (Herman et al., (1996) Proc Natl Acad Sci USA 93, 9821-6; 17) was performed (Table 1 and Methods). As seen in FIG. 4b, FRET, and thereby quenching of QDs, occurred for all genes with IVD, but not with NL nor with the water control when amplified with methylation-specific primers. This highlights that MS-qFRET can be used for reliable, rapid methylation screening and can potentially be valuable for high throughput analyses.

### Example 7

### Ultrasensitive Methylation Detection in Human Sputum Samples

Through our initial analysis, the analytical sensitivity of the MS-qFRET assay to detect very low quantities of methylated DNA (˜15 pg) was illustrated. In order to validate this sensitivity in clinical patient samples with low concentrations of methylated DNA, the methylation status of the ASC/TMS1 in sputum DNA was tested. It is known that ASC/TMS1 protein level is reduced in almost all lung cancer types and hypermethylation of ASC/TMS1 is a marker for late-stage lung cancer, making it a potential predictor of recurrence in patients following surgery for early-stage disease. Methylation of ASC/TMS1 was detected using a nested MSP approach in sputum from patients with stage III or previously resected lung cancer (Machida et al. (2006) Cancer Res 66, 6210-8).

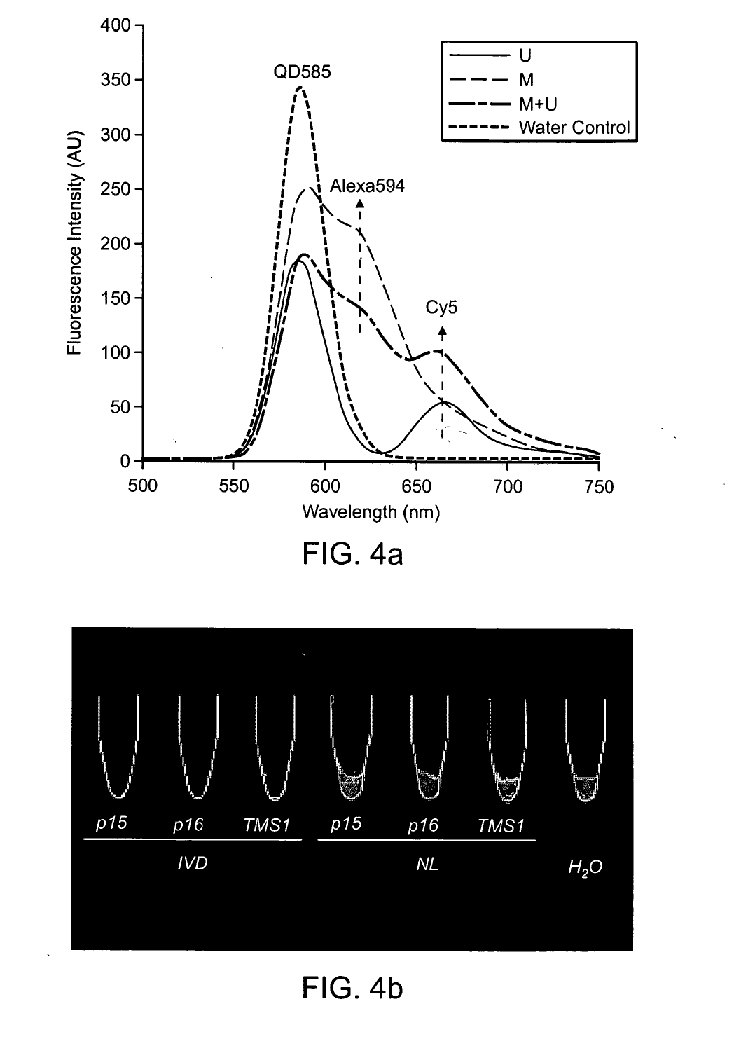

Twenty sputum samples were obtained from individuals with stage III lung cancer. Both standard and nested MSP were used to detect methylation. While only a signal from unmethylated sequences was detected with conventional MSP, the nested assay detected methylated sequences in 3 samples (representative samples shown, FIG. 5A). Comparison of these results to detection with MS-qFRET was performed in a blinded fashion. A representative spectroscopic trace for two patients with differing methylation is shown in FIG. 5B, where a prominent peak seen at 670 nm (Cy5 emission) indicates the presence of methylated ASC/TMS1 promoter. Normalized FRET efficiency (En) (see Methods) for all 20 patients is shown in FIG. 5C, which indicates that the same three samples found to be methylated at ASC/TMS1 by nested MSP were also detected using MS-qFRET. The ability to detect methylation in these clinical samples without the use of a nested approach could make this a promising approach for lung cancer screening.

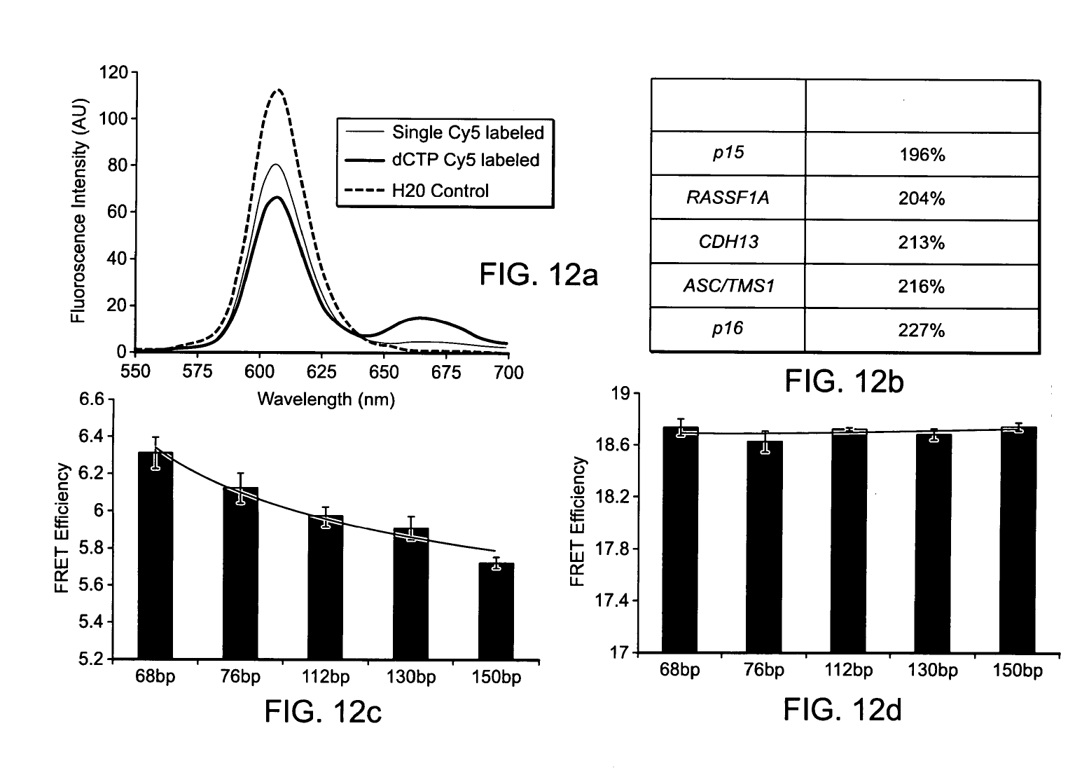

Common approaches to detect gene specific methylation include MSP, nested MSP and real time PCR, which all rely on bisulfite converted DNA (Herman et al., (1996) Proc Natl Acad Sci USA 93, 9821-6; Eads et al. (2000) Nucleic Acids Res 28, E32, Machida et al. (2006) Cancer Res 66, 6210-8). For samples with low concentrations of DNA, nested MSP is frequently utilized, and requires numerous amplification cycles (i.e., greater than 40) (Machida et al. (2006) Cancer Res 66, 6210-8). A drawback to the nested approach is that it can yield false positive results. Additionally, real-time PCR (either SYBR Green or Taqman) offers a quantification method, but is limited by inherent background fluorescence. MS-qFRET overcomes these limitations in a simple endpoint detection format (FIG. 1). The unique optical properties of quantum dots (QD), such as narrow emission bands and large Stokes shift render them ideal FRET donors. This allows minimal fluorescent cross-talk and direct excitation of acceptors (Zhang et al., (2005) Single-quantum-dot-based DNA nanosensor. Nature Materials 4, 826-831, Medintz et al. (2003) Nat Mater 2, 630-8) and permits the design of FRET-based assays with extremely low fluorescent background noise. These features, as well as the high sensitivity of MS-qFRET, enable quantitative methylation detection using substantially reduced PCR amplification cycles as compared to standard techniques (FIGS. 2A and 2B). The ability to detect as little as 15 pg of methylated DNA in unmethylated DNA background underscores the power of MS-qFRET in analyzing challenging samples. By allowing detection with few amplification cycles, MS-qFRET can reduce errors from mispriming, decreases the presence of primer dimers (FIG. 13) and non-specific amplicons, as well as enhances the analysis speed. In addition, use of the nanosensor eliminates the need for nested PCR and adds to the accuracy and precision of methylation detection.

MS-qFRET can be adapted to meet the needs of high throughput screening. The ability to streamline analyses is critical in evaluating large number of samples and can be assisted by a multiplex approach. Specifically, MS-qFRET allows for the use of uniquely labeled fluorophores for the methylated and unmethylated primers within a single tube. This facilitates a more reliable comparison between methylated and unmethylated status for each individual sample as the input DNA is analyzed simultaneously (FIG. 4A). By utilizing other QD donor-acceptor pairs, such as 525QD (max. emission at 525 nm, Quantum Dot Corp.)/Cy3 (max. absorption at 550 nm, max. emission at 570 nm) and 705QD (max. emission at 705 nm, Quantum Dot Corp.)/Cy7 (max. absorption at 743 nm, max. emission at 767 nm), this multiplex reaction can be extended to a multi-gene analysis. QD donor-acceptor pairs of the invention include, but are not limited to, QD525 and BODIPY, QD585 and Alexa594, QD585 and Cy5. Furthermore, in translational applications, a subjective, quick, qualitative screen for methylation may be more powerful than the need for quantification. Direct visual inspection of donor quenching facilitates such a read out (FIG. 4B). Unlike most standard methylation detection techniques that are limited by the capacity of the number of wells or detectors, MS-qFRET can screen thousands of samples at a time using a standard UV scanner. Also, the feature of endpoint detection with a small detection volume renders MS-qFRET compatible with the standard microplate reader and can be straightforwardly implemented in the next-generation 1,536-well format for high-throughput screening. Furthermore, the technology can be extended to quantify methylation by using other standard readouts, such as colorimetric, anything detects fluorescence, or microarray readers. Application of MS-qFRET to cell lines, MDS samples and sputum samples demonstrates utility in a clinical setting. However, the invention is not so limited. The use of MS-qFRET in MDS merely provides proof of concept. The invention provides for the quantification of methylation in virtually any biological sample. By quantifying methylation in cell lines and MDS patient samples, the application of MS-qFRET in monitoring cancer therapy is highlighted (FIG. 3C and FIG. 3D). A common method to assess gene specific response to epigenetic treatment is through gel electrophoresis and therefore is not quantitative. Bisulfite sequencing and MALDI-TOFF are methods that could be used for such screening, but are expensive, time consuming and may not be universally accessible. One advantage to MS-qFRET is easy adoption into current MSP methodology. Additionally, by assigning values through a q-score MS-qFRET allows for a greater resolving capability in monitoring methylation reversal by being more sensitive and quantitative (FIG. 3). The ability to use MS-qFRET for methylation detection in sputum samples (FIG. 5) highlights the feasibility for its future application in routine processing of patient samples with low amounts of DNA such as serum, stool and urine. Based on prior studies (Machida et al. (2006) Cancer Res 66, 6210-8), DNA concentration in the sputum samples was estimated to vary from 30 pg/μL to 200 pg/μL. Clear demonstration of methylation in patient sputum is observed when compared to the background noise using MS-qFRET (FIG. 5B). MSP and real-time PCR both fall short of direct detection of methylation in such samples.

The numerous attributes of QD-FRET nanosensors make them ideal for detecting and quantifying methylation. MS-qFRET is cost-effective for quantification of DNA methylation as it does not require the expensive setup necessary for real-time PCR and pyrosequencing (Eads et al. (2000) Nucleic Acids Res 28, E32 34). In addition, MS-qFRET is fully compatible with standard MSP (Herman et al., (1996) Proc Natl Acad Sci USA 93, 9821-6), and significantly transforms this most widely used technology for methylation detection to become a quantitative, high-throughput and ultrasensitive format via the end-labeling of existing MSP primers and the inclusion of off-the-shelf QDs for fluorescent measurements. Hence, MS-qFRET is a method that can be readily adopted by a broad range of laboratories and will likely have an immediate impact on basic and clinical research.

### Example 8

### DNA Extraction

Genomic DNA samples were obtained from various sources. The following protocol was demonstrated using whole blood. Human blood samples were collected from volunteers with fully informed consent. All the chemicals were purchased from Sigma Aldrich Inc. unless otherwise stated. A list of buffers used is summarized in Table 2.

| MOB Buffers | Reagents |
|---|---|
| Lysing Buffer | Protease K |
| MOB Binding Buffer | 8M Sodium Hyperchlorate |
| IPA | 100% Isopropanol |
| Magnetic Beads | silica superparamagnetic particles |
| MOB Wash 1 | 37.5 ml 100% IPA |
| | 12.5 ml 8M Hypercholate |
| MOB Wash 2 | 75% Ethanol |
| Bisulfite Conversion Buffer | 15 μl .001M Hydroquinone |
| | 250 μl 6.9M Sodium Bisulfite |
| | 5.5 μl 2M Sodium Hydroxide |
| MOB Elution Buffer | 10 mM Tris pH 8 |

Blood samples were stored with EDTA to prevent coagulation. 20 μL 0.5 mg/ml of protease K (Invitrogen) was added to a 1.5 mL microcentrifuge tube, followed by 100 μL whole blood sample. Binding buffer (8M NaClO₄) was prepared by dissolving 49 g of sodium hypercholorate in 50 mL water. The solution was vortexed vigorously till the salt fully dissolved. 100 μL of binding buffer was added to the sample and mixed by reverse pipetting. The microcentrifuge tube was incubated at 70° C. for 10 minutes to lyse the cell. After incubation, 100 μL of 100% Isopropanol Alcohol (IPA) and 30 μL silica superparamagnetic particles (SSP) (Qiagen Corporation) was added followed by reverse pipetting. The tube was then incubated at room temperature (25° C.) for 10 minutes.

SSP were immobilized by placing the microcentrifuge tube on a Magnetic Particle Concentrator (MPC) (Invitrogen Corp.). The solution was drawn from the immobilized SSP while the microcentrifuge tube was on the MPC. The immobilized SSP pellet was then washed once by adding 350 μL Washing Buffer 1 (WB1) (75% IPA+2M Sodium Hyperchlorate) and resuspending the pellet in the solution. The pelleted SSP were washed twice more with 250 μL Washing Buffer 2 (WB2) (75% ethanol). After the second wash, the solution was completely removed, leaving only the immobilized SSP in the microcentrifuge tube. The DNA was fixed on the SSP and ready for subsequent bisulfite treatment.

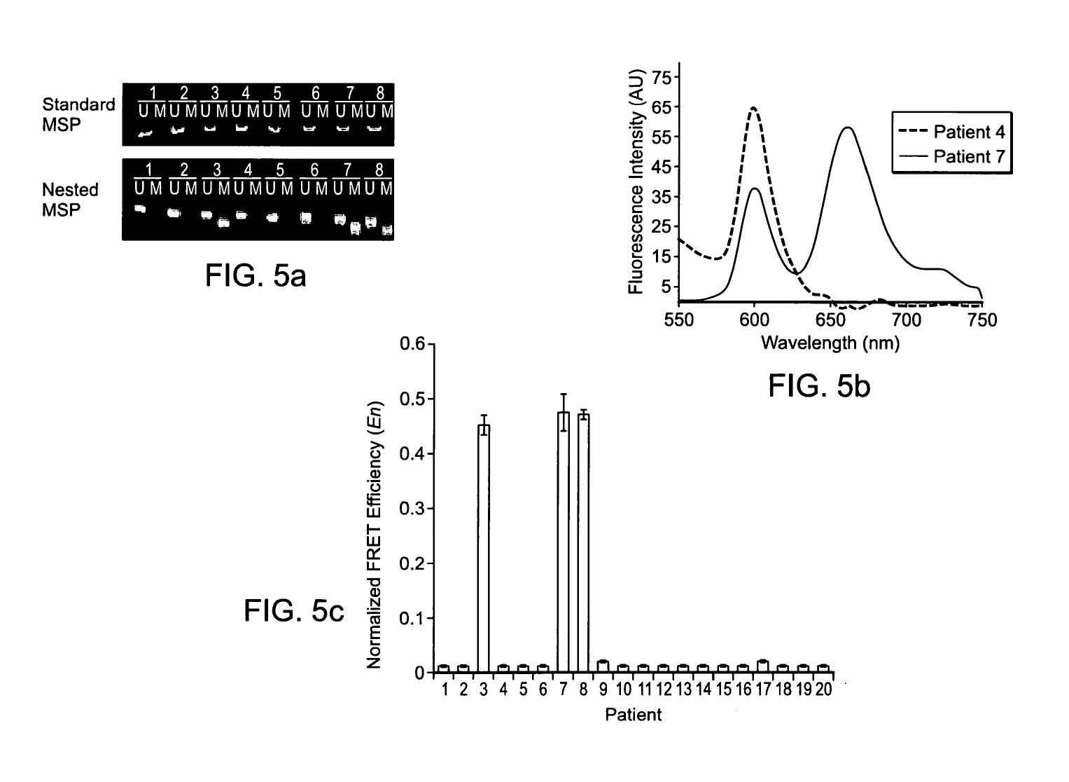

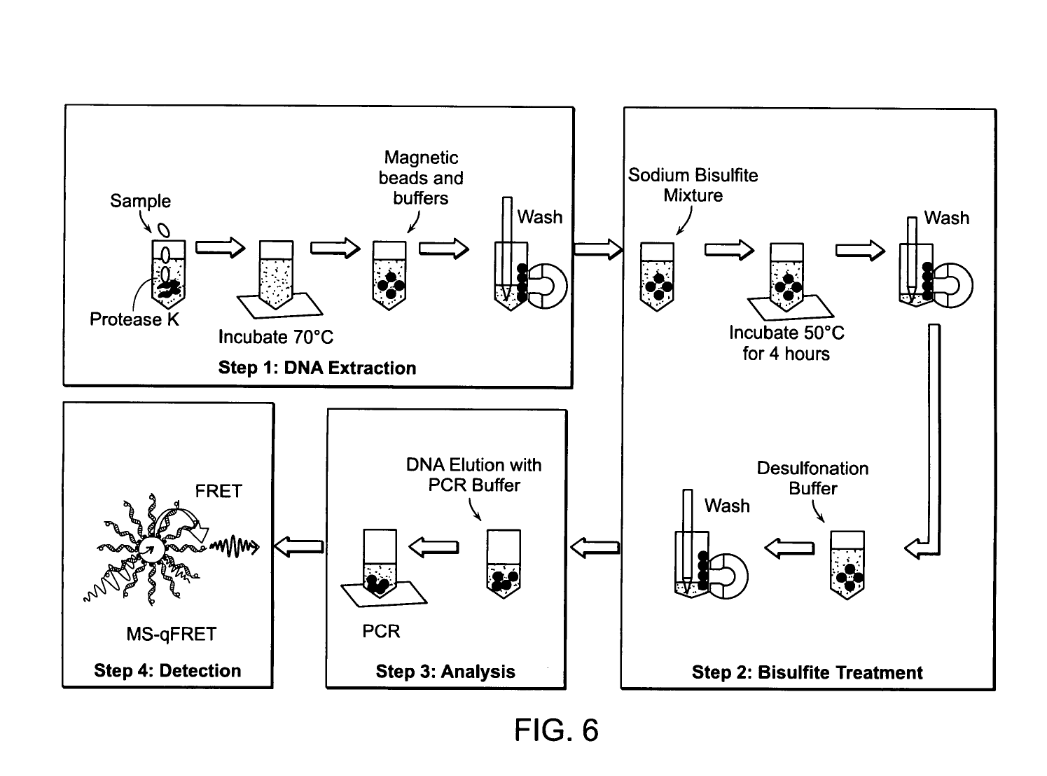

Details of MOB extraction has been illustrated in FIG. 6. In order to assess the performance of MOB extraction, 12 tubes of 200 μL whole blood were obtained. Tubes were then split into aliquots of 100 μL in order to avoid bias from differences in cell counts in the buffy coat and supernatant. MOB was used on 12 tubes while ethanol precipitation was used for the remaining 12. Measurements were made at this point to determine the yield of the DNA. A side-by-side comparison was made for the same sample using standard ethanol precipitation for DNA extraction. Results obtained are presented in FIG. 7A. For each of the 12 tubes, total DNA yield after DNA extraction using SSPs was over 3500% to 7000% more than ethanol precipitation. Measurements of DNA concentration are made by using the Nanodrop 3000 (Nanodrop Technologies). FIG. 7B presents a comparison of the average concentration of DNA obtained per tube. The average concentration through MOB was 75 ng/μL when compared to an average of 5 ng/μL through column extraction. A significantly higher concentration allows for a greater number of genes to be screened for methylation. In addition, rare events through circulating tumor DNA that could possibly be missed may be picked up through MOB extraction.

### Example 9

### Bisulfite Treatment

50 μL of water and 5.5 μL of 2M NaOH were added to the microcentrifuge tube to resuspend the pelleted SSP. The resuspended SSP were incubated at 37° C. for 10 minutes. Hydroquinone solution was prepared by dissolving 0.11 g hydroquinone in 50 mL dH₂O. 15 μL of prepared hydroquinone solution was added to the microcentrifuge tube. Sodium bisulfite (NaBst) solution was made by adding 36 g of NaBst to 50 mL dH₂O. The solution was saturated, it was normal for not all of to be dissolved. Before adding 250 μL of the prepared NaBst solution to microcentrifuge tube, the solution was homogenized by reverse pipetting for 15 seconds. The microcentrifuge tube was gently vortexed and incubated at 50° C. for at least about 4 hours. 200 μL of binding buffer was added to the microcentrifuge tube together with 200 μL IPA. The sample was then mixed by pipetting the microcentrifuge tube for 10 seconds followed by incubation at room temperature for 10 minutes. After incubation, the microcentrifuge tube was placed on the MPC to pellet the SSP. The solution was removed from the immobilized SSP and discarded. 200 μL of WB1 was added to resuspend the SSP pellet. The SSP was again pelleted with the MPC and the solution was discarded. The washing step was repeated twice with WB2. After the second wash, the solution was removed, leaving the SSP immobilized in the microcentrifuge tube. 50 μL of dH₂O and 5.5 μL 2M NaOH were added to the SSP. The pelleted SSPs were resuspended in the solution and incubated at room temperature (25° C.) for 10 minutes. 200 μL of binding buffer was then added to microcentrifuge tube followed by reverse pipetting. The SSP were pelleted by placing the microcentrifuge tube on the MPC. The solution was separated from the immobilized SSP. The SSP were then washed 3 times with 200 μl, WB1 and 125 μL WB2 as described previously.

After the third wash, the supernatant solution was removed after the SSP were pelleted and immobilized in the microcentrifuge tube. The bisulfite converted DNA, which were fixed on the SSP, were either eluted in 10 mM Tris at 70° C. and stored or directly subject to MSP together with the SSP by eluting with standard MSP PCR Buffer (Herman et al., (1996) Proc Natl Acad Sci USA, 93, 9821-9826).

DNA concentrations are measured again at the end of bisulfite treatment using the Nanodrop 3000. The 12 tubes from which DNA had been extracted through ethanol precipitation were now subject to 2 methods of bisulfite treatment: a commercially available column-based kit (Zymo) and the standard bisulfite treatment protocol (Herman (1996) Proc Natl Acad Sci USA, 93, 9821-9826.). Care was taken to have total 2 μg input DNA for purposes of comparing efficiency in recovering DNA for the three methods. FIG. 7C compares the recovery of DNA in the three methods. Average recovery was 79.58% by MOB bisulfite treatment while recovery from column and standard treatment was 14.41% and 19.64% respectively. By having an average recovery 4.67 times greater than traditional methods, MOB bisulfite treatment allows for efficient recovery that once again facilitates greater discovery by allowing more input DNA for a more genes to be analyzed.

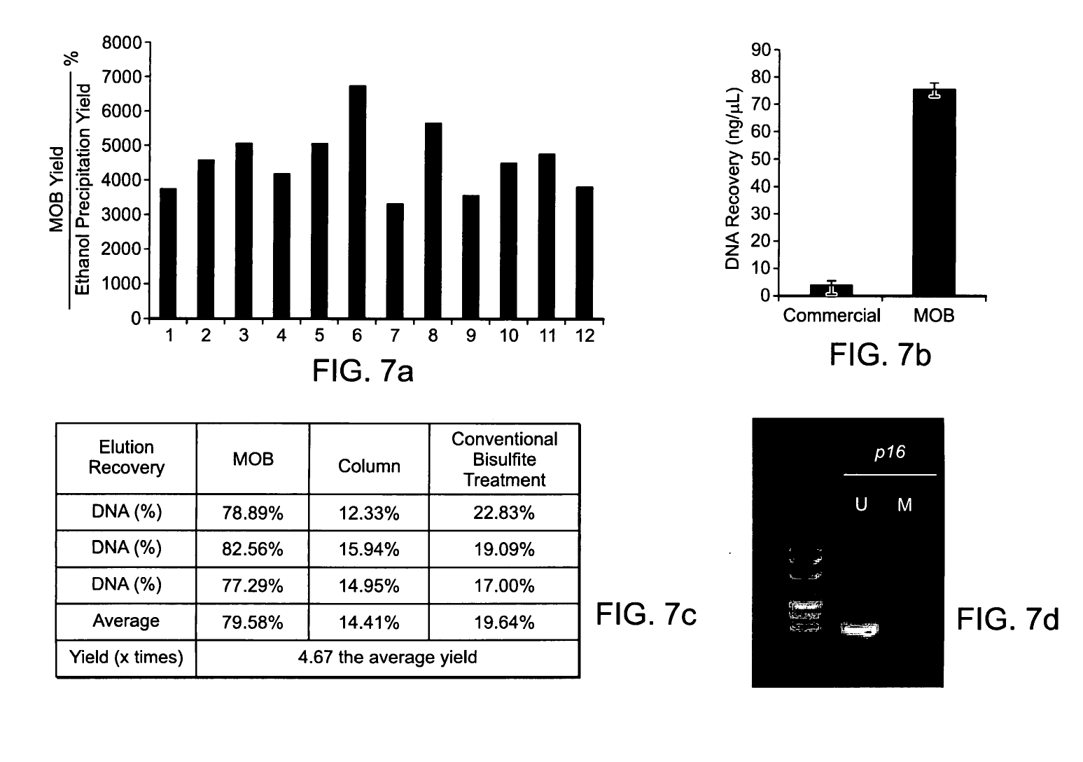

Separately, the reaction was downscaled 10 fold and as little as 10 μL of whole blood was used as input. MOB extraction and bisulfite conversion was carried out in a PCR tube. Upon eluting DNA with PCR buffer, MSP with p16 primers was performed to determine if bisulfite conversion was successful. As shown in FIG. 7D, a strong unmethylated band was observed demonstrating that SPP did not hinder bisulfite conversion or the MSP reaction. In order to determine whether the efficiency of conversion was comparable to standard methods, a real time PCR assay was setup with input DNA from bisulfite treated DNA from both methods. 3 triplicate reactions were setup with input DNA being from varying bisulfite treatment hours (0 hrs, 4 hrs, 8 hrs and control (16 hrs of conventional bisulfite treatment). FIG. 8 summarizes results from the real time PCR and demonstrates that the efficiency of bisulfite conversion is comparable in both methods. Results also indicate that as little as 4 hours of bisulfite treatment is sufficient to generate efficiency conversion through MOB.

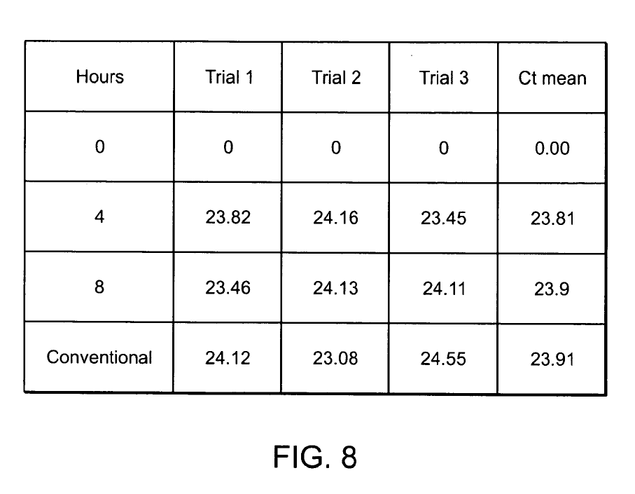

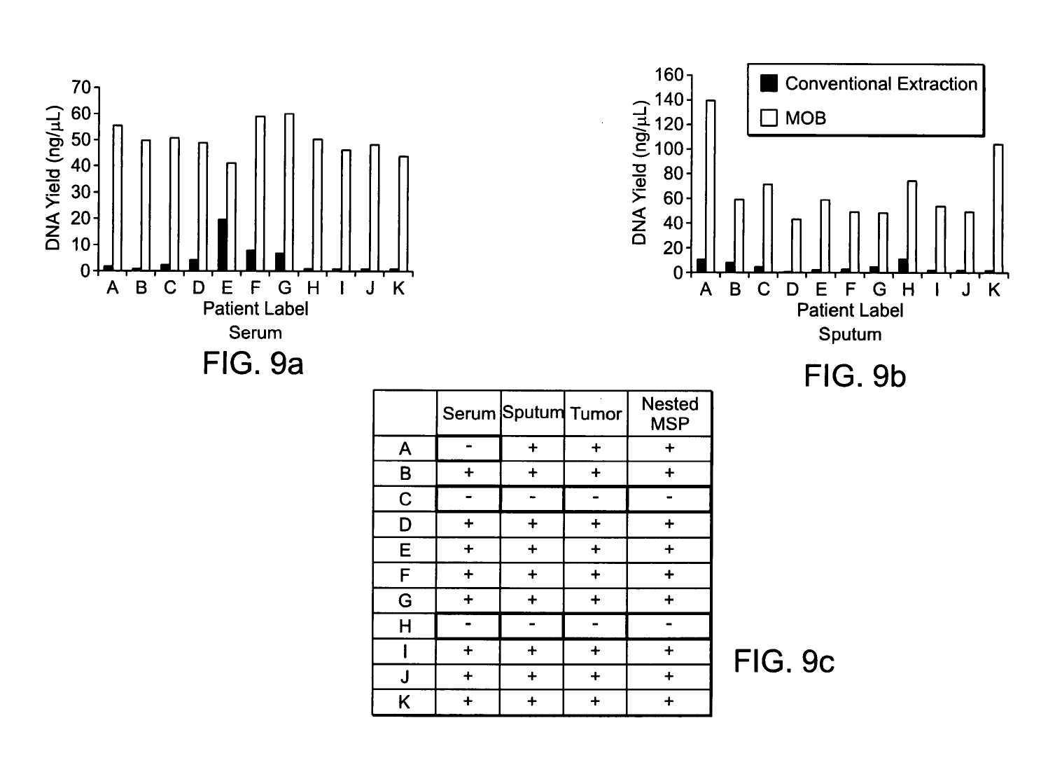

Having successfully demonstrated extraction, bisulfite treatment and methylation detection in whole blood, the technique was extended to serum and sputum samples from patients. Using conventional techniques, DNA yield from serum ranged from 0.2 to 0.5 μg from a 750 μL sample. This corresponded with prior literature which also reported average serum DNA yields of 39 ng/mL from 10 mL plasma (Belinsky et al., (2002) Journal of Clinical Ligand Assay, 25, 95-99). Due to a limited supply of patient serum, the need for large amounts of serum restricted the number of genes that could be tested for methylation. Alternatively, serum methylation has been detected from as little as 200 μL using carrier DNA in addition to nested MSP. As seen in FIG. 9A, through MOB, methylation has been detected in as little as 200 μL of serum while maintaining an average yield of 40 to 70 ng/μL.

Based on these results, MOB has completely eliminated the need for large amounts of serum as enough DNA for over 400 genes can be facilitated from as little as 200 μL serum, assuming that each MSP reaction requires an input of 20 ng DNA. Similar results were observed when DNA was extracted from sputum (FIG. 9B). MOB allows for an increased yield of DNA due to the strong affinity of the magnetic beads for DNA, allowing for single tube extraction, purification, bisulfite treatment, and MSP. Utilizing the change of affinity in variant buffers, large amounts of purified DNA can then be eluded by our elution buffer. This technique greatly increases yield of DNA for methylation detection, which is especially important for sputum and serum samples. Upon analyzing the methylation status of serum and sputum samples with DNA extracted from tumor samples, results coincided in across the board for methylation in p16 promoter for 11/12 patients (FIG. 9C). For the one patient anomaly, promoter methylation results in sputum and tumor coincided, while serum results showed the promoter was unmethylated. A possible reasoning for this could be the absence of circulating tumor DNA for this patient.

In sum, as reported herein, the invention provides a novel and improved technique for DNA extraction, bisulfite treatment and methylation detection in a single tube using SSP as a solid substrate for DNA manipulation. The demonstrated technique has a significantly larger yield after both DNA extraction as well as bisulfite treatment when compared to conventional methods. In addition, MOB has great potential for automation and is significantly faster than current methods. MOB is a method that can be readily adopted by a broad range of laboratories and will likely have an immediate impact on basic and clinical research.

### Example 10

### Enhancing Quantum Dot FRET

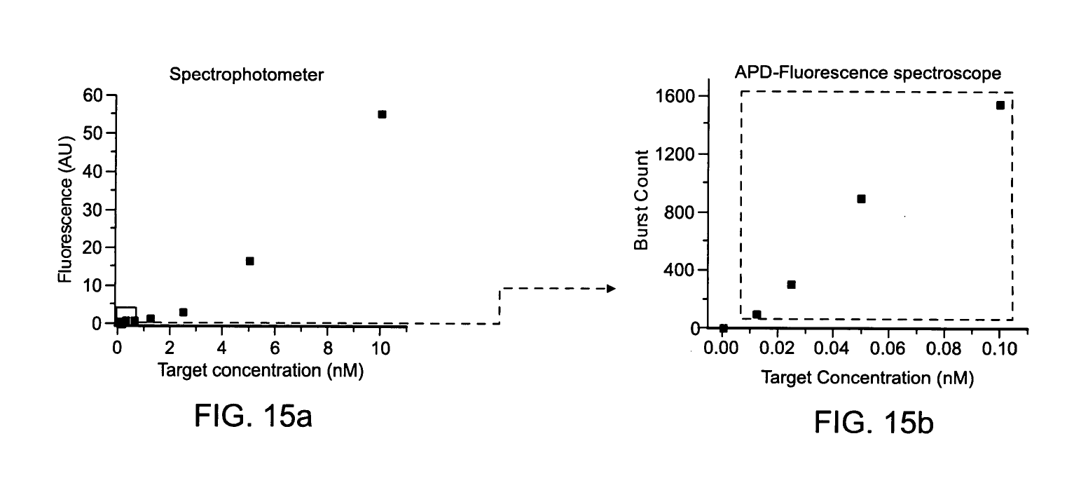

A unique advantage of QD-FRET is that its energy transfer efficiency can be enhanced by increasing the acceptor:donor ratio in the system. Accordingly, the invention provides for the use of fluorophore (CyDye) labeled nucleotides in PCR to generate multiple CyDye labeled PCR products for conjugation with QD, amplifying FRET signal and the sensitivity of MS-QFRET. The enhancement of sensitivity will also facilitate quantification of DNA methylation by endpoint detection with further reduction in amplification, leading to an additional increase in analysis speed, dynamic range, and accuracy. Moreover, as shown in (FIG. 15), the extremely low intrinsic fluorescence background of QD-FRET leads to a condition that the overall sensitivity of the MS-QFRET system may be limited by the optical sensitivity of the fluorescence reader. In order to best demonstrate the ultimate sensitivity of MS-QFRET, the invention employs a highly sensitive and user-friendly FRET detection system coupled with avalanche photodiodes (APDs) for use with MS-QFRET. To further enhance the throughput of MS-qFRET, the FRET detection unit will be implemented to be capable of simultaneously measuring 96 and 384 samples. A data acquisition and processing program will also be developed for automatic FRET analysis and determination of methylation status.

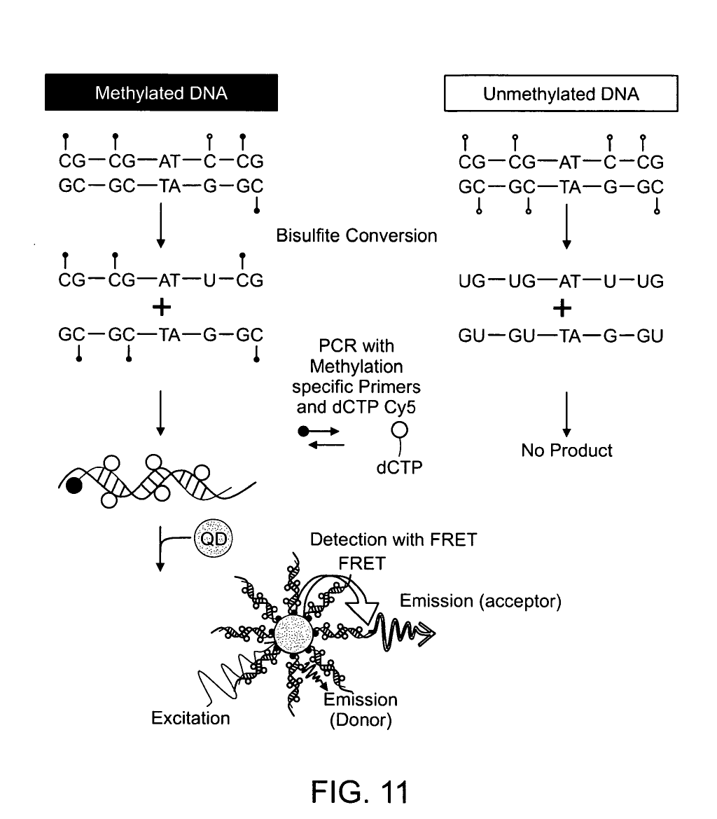

To amplify QD-FRET signals by producing multiple-labeled PCR products, the ultimate sensitivity of a detection system is usually characterized by its signal-to-noise ratio. For a FRET system, the signal level (the intensity of acceptor fluorescence emission) is dependent on the energy transfer efficiency, the quantum yield, and the photostability of the acceptors, while the noise level (background fluorescence intensity) is dependent on the degrees of both the leakage of donor fluorescence emission to the acceptor emission wavelength region and the direct excitation of acceptor. One unique feature of the QD-FRET system is that the FRET efficiency can be enhanced by increasing the number of fluorophore acceptors associated to a QD donor. For MS-qFRET assays, one way to increase the acceptor:donor ratio is to reduce the quantity of QDs added to capture the amplicons. However, the reduction of QDs may in turn lead to a decrease in the overall fluorescence intensity (for both acceptors and acceptors). In addition, the possible deficit of QDs for conjugation with amplicons may complicate the quantification of DNA targets. To take advantage of signal amplification by increasing the acceptor:donor ratio without the need for reducing the QD quantity, the invention provides for the use of labeled nucleotides in the PCR reaction to facilitate generation of PCR amplicons that are labeled with multiple fluorophores/acceptors (FIGS. 12A and 12B. An additional benefit of using CyDye-conjugated nucleotides is that it may eliminate the need of using CyDye-labeled primer, reducing the cost for primer preparation for MS-QFRET. In particular embodiments, CyDyes (Cy3, Cy3.5, Cy5 or Cy7) are used for the labeling. To minimize the self-quenching effect, a mixture of unlabeled dNTPs may be included with the labeled nucleotides, CyDye-dCTP (Amershan) at a mixing ratio of ˜2:1 to 8:1, as suggested by the vendor in the PCR process. Different mixing ratios will be tested to determine the optimal one that produces the maximal QD-FRET-mediated acceptor signals. The PCR protocol will also be optimized to maximize the synthesis efficiency.

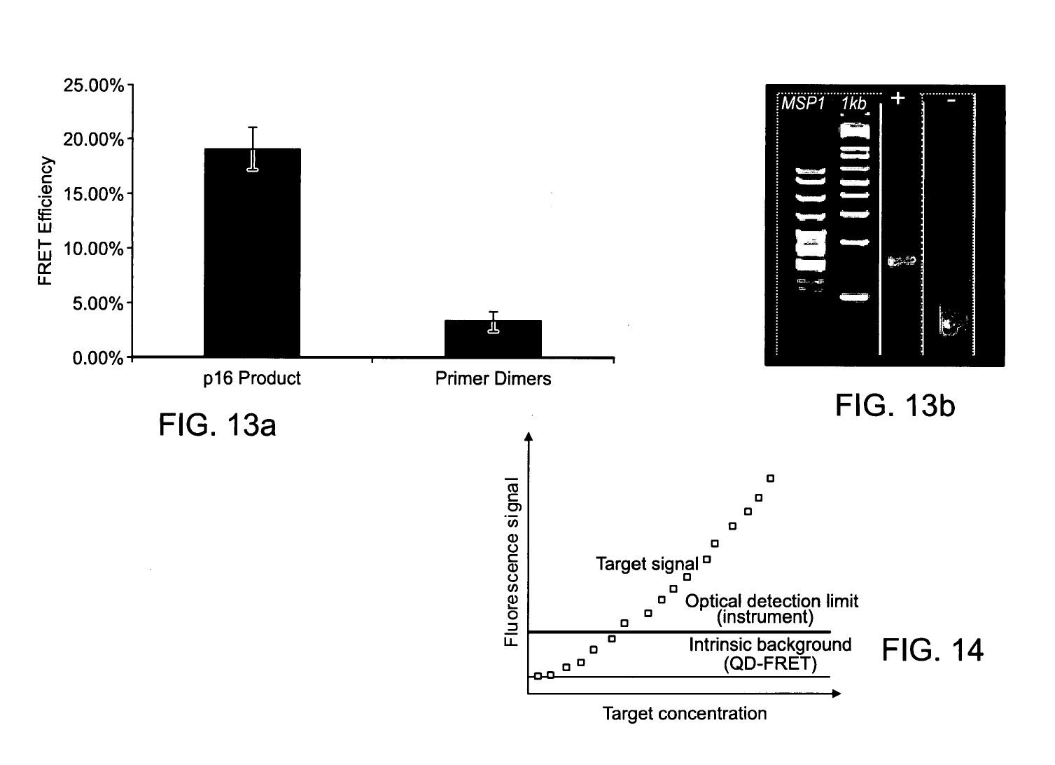

The characteristic of simple endpoint detection of MS-qFRET renders it amenable to a variety of fluorescence readouts, such as spectrophotometer, microplate readers, or microarray readers for analysis of DNA methylation. Even with the favorable feature of the extremely low fluorescence background level of the QD-FRET technology, true signal may not be resolvable due to the poor optical sensitivity of the reader, as illustrated in FIG. 15. Indeed, when detecting control samples from mixtures of DNA (Cy5 labeled and biotinylated) and 605QD with the spectrophotometer (Nanodrop 3300), QD-FRET induced Cy5 signal was only detected down to ˜1 nM target concentration. In contrast, Cy5 signals from further diluted samples were still able to be detected by a fluorescence spectroscope equipped with an avalanche photodiode (APD) (FIG. 14). This result suggests that the overall sensitivity of MS-QFRET will be better presented by using a detector with a higher optical sensitivity.

The results reported herein were carried out using the following methods and materials.

### DNA Isolation and Bisulfite Modification.

In vitro methylated DNA (IVD) was obtained from treatment of leukocyte DNA with SSSI methyltransferase. Peripheral blood lymphocytes (NL) were isolated from blood from normal volunteers, sputum samples were obtained from patients with a known smoking history, and bone marrow aspirate samples were obtained from MDS patients. All samples were obtained after informed consent and IRB approval of the clinical studies. RKO cells were cultured and treated with 1 μM DAC and collected at fixed time points. DNA extraction and bisulfite modification was performed as previously described (Herman et al., (1996) Proc Natl Acad Sci USA 93, 9821-6).

### Primers.

The primer sequences used in Examples 1-7 are described in Table 1 and have been previously validated (Herman et al., (1996) Proc Natl Acad Sci USA 93, 9821-6, Machida et al. (2006) Cancer Res 66, 6210-8). The primers for MS-qFRET were replicates of those used in standard MSP except for 5′ labeling of the forward primer with biotin and the reverse primer with an organic fluorophore (Integrated DNA Technologies (IDT)). Labeled primers were HPLC purified.

### MSP and Nested MSP.

The MSP reaction consists of a mixture of 3 μl of target DNA added to 22 μl of reaction buffer containing 10×PCR buffer (16.6 mM ammonium sulfate/67 mM Tris, pH 8.8/6.7 mM MgCl₂/10 nM 2-mercaptoethanol), dNTPs (Continental Lab Products, each at 1.25 mM), MSP primers from the gene of interest (300 ng each per reaction), and 1 μl of HotStart Taq polymerase (Qiagen Corporation). For p16, conditions were 95° C. for 15 minutes, followed by 35 cycles of 30 seconds at 95° C., 30 seconds at the annealing temperature 64° C. and 30 seconds at 72° C., with a final extension cycle of 72° C. for 5 minutes. Annealing temperature was 58° C. for p15 and TMS1. Unmethylated (NL), Methylated controls (IVD) and water controls (no template added) were utilized. All PCR products (5 μl) were loaded onto a 2% agarose gel, stained with GelStar (Lonza) and directly visualized under UV-illumination.

The nested MSP procedure required 2 PCR reactions and was performed as previously described (Machida et al. (2006) Cancer Res 66, 6210-8).

### MS-qFRET.

### General Procedure.

PCR with labeled primers was run as previously described. Products were then subject to PCR purification (Qiagen Corporation) in order to recover PCR product that is free of primers, primer-dimers, Taq and dNTPs. For conjugating with quantum dots (Invitrogen Corporation), 1 μL of 100 mM NaCl is mixed with 7 μL PCR mix. 1 μL of deionized (DI) H₂O is added to this mix. Finally, 1 μL of 1 nM QD is added and the mixture is left undisturbed for 15 minutes.

### Quantitative Analysis.

Mixtures of defined methylation levels range from 100%, 75%, 50%, 25%, and 1% of the total 150 ng input DNA were obtained by varying quantities of IVD diluted in a background of NL. The mix was used as the input template for a low-amplification (16 cycles) MSP reaction with labeled p16 primers. To quantify the level of methylation, a “q-score”: a score that is based on the normalized FRET efficiencies of acceptor and donor emission in MS-qFRET was defined. In any FRET process, as the level of the acceptor emission increases, the decay of donor emission increases as well. The FRET efficiency can then be calculated based on the proximity ratio formalism,

$$E = \frac{{I}_{A}}{{I}_{A} + {I}_{D}}$$

(I_{D} and I_{A} corresponding to donor and acceptor intensity). Further, the q-score was determined by normalizing the calculated E for the DNA mixture to an appropriate concentration of IVD only as a methylated control (q-score=1) and NL only and as an unmethylated control (q-score=0). By including positive and negative controls in every assay a standard curve was created in order to quantify and compare methylation levels of unknown samples using low-amplification cycles.

### Ultra-Sensitive Screening.

150 ng IVD was serially diluted to 1:10,000 and mixed with a background of NL (150 ng). MSP with labeled p16 primers was run for 40 cycles. For analysis, QD concentration was at 5 pM. Upon QD605 conjugation, confocal spectroscopy (Zhang et al., (2005) Single-quantum-dot-based DNA nanosensor. Nature Materials 4, 826-831) was used to observe Cy5 peaks against the inherent low background. Peak counts were collected from Cy5 fluorescence bursts from methylated targets attached to single QDs passing through the focal volume. The data was processed and peaks were counted and normalized using a custom LabView program.

### Determination Through Visualization.

100 ng of IVD and NL was used as input DNA in a MSP reaction with p16, p15 and ASC/TMS1 primers for 35 cycles. QD605 concentration was maintained at 2 nM for both conjugation and analysis. Analysis was made through visualization under a UV-lamp.

### Multiplexed Analysis.

p16 methylated reverse primer was labeled with Alexa594 while the unmethylated reverse primer was labeled with a Cy5 fluorophore. 50 ng methylated target (IVD) and 75 ng unmethylated target (NL) were each individually mixed with both unmethylated and methylated primers. A mixture of 35% methylated and 65% unmethylated target (125 ng total DNA) was also subject to the same PCR conditions and primers. 35 cycles of MSP was run with conditions as previously described. Finally, QD585 at 1 nM was used during conjugation and analysis.

### Instrumentation.

Fluorescence measurements for MS-qFRET were made using NanoDrop 3300 (NanoDrop). Using this setup, fluorescence readouts with as little as 2 μL of sample were measured. The confocal spectroscope, sensitive at the level of single-molecule fluorescence, was setup and used as previously described (Zhang et al., (2005) Single-quantum-dot-based DNA nanosensor. Nature Materials 4, 826-831).

dCTP cy5 Labeled PCR

60 μM of commercially available dCTP Cy5 (Amersham Technologies) was used with a mixture of 100 μM of dATP, dDTP, dGTP and 60 μM dCTP. MSP reaction setup was as described herein. The resulting product was purified using Qu9iagen Nucleotide Purification kit to remove free nucleotides and excess primers.

Hanger Probe Labeled

PCR product was quantified for concentration using A260/A280 ratio. After determining the concentration of PCR product, a corresponding Cy5 labeled probe was selected and diluted to 10 times the PCR product concentration. The PCR product was mixed with the probe and placed at 95 C for 5 minutes. The mixture was cooled to room temperature and incubated at 20 C overnight. For conjugating with QD605, 5 μl of 100 mM NaCl was mixed with 3 μl PCR-hanger probe mix. 1 μl of deionized water was added to the mix. Finally, 1 μl of 1 nM AD605 is added and the mixture was incubated for 15 minutes.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for detection of polynucleotide methylation, the method comprising
(a) amplifying a polynucleotide comprising unmethylated cytosines converted to uracil with a primer pair, wherein one primer comprises a binding moiety having affinity for a binding partner, to obtain an amplicon;
(b) capturing the amplicon comprising the binding moiety with a binding partner fixed to a quantum dot; and
(c) inducing fluorescence resonance energy transfer between the quantum dot and the detectable label, thereby detecting polynucleotide methylation.

2. A method for quantification of polynucleotide methylation, the method comprising
(a) amplifying a polynucleotide comprising unmethylated cytosines converted to uracil with a primer pair, wherein one primer comprises a binding moiety having affinity for a binding partner, to obtain an amplicon;
(b) capturing the amplicon comprising the binding moiety with a binding partner fixed to a quantum dot; and
(c) inducing fluorescence resonance energy transfer between the quantum dot and the detectable label, thereby detecting polynucleotide methylation.

3. The method of claim 1 or 2, wherein a second primer of the pair comprises a detectable label.

4. The method of claim 1 or 2, wherein the amplicon is detectably labeled by hybridization with a detectable probe.

5. The method of claim 1 or 2, wherein the amplicon is detectably labeled by incorporation of a detectably labeled nucleoside.

6. A method for detection of polynucleotide methylation, the method comprising
(a) amplifying a polynucleotide comprising unmethylated cytosines converted to uracil with a primer pair, wherein one primer comprises a binding moiety having affinity for a binding partner and the other primer comprises a detectable moiety, to obtain an amplicon;
(b) capturing the amplicon comprising a binding moiety and a detectable label with a binding partner fixed to a quantum dot; and
(c) inducing fluorescence resonance energy transfer between the quantum dot and the detectable label, thereby detecting polynucleotide methylation.

7. A method for detection of polynucleotide methylation, the method comprising
(a) amplifying a polynucleotide comprising unmethylated cytosines converted to uracil with a primer pair, wherein one primer comprises a binding moiety having affinity for a binding partner, and the amplification is carried out using at least one detectably labeled base;
(b) capturing a labeled-amplicon with a binding partner fixed to a quantum dot; and
(c) inducing fluorescence resonance energy transfer between the quantum dot and the detectable label, thereby detecting polynucleotide methylation.

8. A method for detection of polynucleotide methylation,
(a) amplifying a polynucleotide comprising unmethylated cytosines converted to uracil with a primer pair, wherein one primer comprises a binding moiety having affinity for a binding partner, to obtain an amplicon;
(b) denaturing the amplicon and hybridizing the amplicon with a detectably labeled probe to detectably label the amplicon;
(c) capturing the labeled-amplicon with a binding partner fixed to a quantum dot; and
(d) inducing fluorescence resonance energy transfer between the quantum dot and the detectable moiety, thereby detecting polynucleotide methylation.

9. The method of any of claims 1-8, wherein the binding moiety is a group that mediates ligand binding or a chemically reactive group.

10. The method of any of claims 1-8, wherein the chemically reactive group is an amine, carboxyl, aldehyde, or sulfhydral groups

11. The method of any of claims 1-8, wherein the binding moiety and binding partner are biotin/streptavidin, antibody/antigen, or amine-succinimidyl ester.

12. A method for detection of DNA methylation, the method comprising
(a) contacting DNA with a reagent that converts unmethylated cytosines to uracil;
(b) amplifying the DNA using forward and reverse primers, wherein one primer is labeled with a binding moiety and the other is labeled with a fluorophore;
(c) capturing a labeled amplicon using a quantum dot comprising a binding partner having affinity for the binding moiety; and
(d) exciting fluorescence resonance energy transfer between the quantum dot and the fluorophore and detecting fluorophore emission, thereby detecting DNA methylation.

13. A method for detection of DNA methylation, the method comprising
(a) contacting DNA with sodium bisulfite under conditions that provide for the conversion of unmethylated cytosines to uracil;
(b) amplifying the DNA using forward and reverse primers, wherein one primer is labeled with biotin and the other is labeled with a fluorophore;
(c) capturing the labeled amplicon using a quantum dot comprising streptavidin; and
(d) exciting fluorescence resonance energy transfer between the quantum dot donor and the fluorophore acceptor and detecting fluorophore emission, thereby detecting DNA methylation.

14. The method of claim 13, wherein the fluorophore emission occurs concurrently with quantum dot quenching.

15. The method of any of claims 1-13, wherein the polynucleotide is obtained from a biological sample.

16. The method of claim 15, wherein the biological sample is selected from the group consisting of sputum, stool, blood, blood serum, plasma, cerebrospinal fluid, urine, seminal fluids, ejaculate, and vaginal secretions.

17. The method of any of claims 1-13, wherein the method detects an alteration in promoter methylation level relative to a reference.

18. A method for diagnosing or characterizing a disease, the method comprising
(a) contacting DNA extracted from a biological sample with sodium bisulfite under conditions that provide for the conversion of unmethylated cytosines to uracil;
(b) amplifying the DNA using forward and reverse primers, wherein one primer is labeled with biotin and the other is labeled with a fluorophore;
(c) capturing a labeled amplicon comprising biotin and fluorphore using a quantum dot comprising streptavidin; and
(d) exciting fluorescence resonance energy transfer between the quantum dot donor and the fluorophore acceptor and detecting fluorophore emission;
(e) comparing said fluorophore emission with a reference, wherein detection of an alteration in DNA methylation diagnoses or characterizes a disease.

19. A method for diagnosing a neoplasia, the method comprising
(a) contacting DNA extracted from a biological sample with sodium bisulfite under conditions that provide for the conversion of unmethylated cytosines to uracil;
(b) amplifying the DNA using forward and reverse primers, wherein one primer is labeled with biotin and the other is labeled with a fluorophore;
(c) capturing the labeled amplicon using a quantum dot comprising streptavidin; and
(d) exciting fluorescence resonance energy transfer between the quantum dot donor and the fluorophore acceptor and detecting fluorophore emission, thereby identifying a neoplasia.

20. A method for monitoring a disease characterized by an alteration in DNA methylation, the method comprising
(a) contacting DNA extracted from a biological sample with sodium bisulfite under conditions permissive for the conversion of unmethylated cytosines to uracil;
(b) amplifying the DNA using forward and reverse primers, wherein one primer is labeled with biotin and the other is labeled with a fluorophore;
(c) capturing the labeled amplicon using a quantum dot comprising streptavidin; and
(d) exciting fluorescence resonance energy transfer between the quantum dot donor and the fluorophore acceptor and detecting fluorophore emission;
(e) comparing said fluorophore emission with a reference.

21. The method of any of claims 1-20, wherein the alteration is an increase or a decrease in methylation.

22. The method of any of claims 1-20, wherein the method detects a neoplasia in a subject.

23. The method of any of claims 1-20, wherein the method detects or characterizes methylation status of lung cancer, acute myeloid leukemia, or myelodysplastic syndrome.

24. The method of any of claims 1-20, wherein the method characterizes prognosis of a subject having an alteration in methylation.

25. The method of any of claims 1-20, wherein the method monitors a tumor.

26. The method of any of claims 1-20, wherein the method monitors a tumor's responsiveness to therapy.

27. The method of any of claims 1-20, wherein the method detects as little as 5, 10, 15 or 20 pg of methylated DNA in the presence of an excess of unmethylated alleles.

28. The method of any of claims 1-20, wherein the method detects methylated DNA after as few as 5, 8, 10, or 12 PCR cycles.

29. The method of any of claims 1-20, wherein the method provides for quantitative endpoint detection of methylation.

30. The method of any of claims 1-20, wherein the method detects methylation status in a polynucleotide isolated from as few as 3-5 cells.

31. The method of any of claims 1-20, wherein the method provides for detection of a single quantum dot or a single methylated molecule.

32. The method of any of claims 1-20, wherein the method detects DNA methylation in a biological sample obtained from a subject having or at risk of developing lung cancer or myelodysplastic syndrome.

33. The method of any of claims 1-20, wherein the method provides for multiplex analyses.

34. The method of any of claims 1-20, wherein the method further comprises amplifying DNA using a second pair of primers, at least one of which comprises a fluorophore that is distinguishable from the fluorophore present on the first set of primers.

35. The method of any of claims 1-19, wherein the method provides for the concurrent analysis of unmethylated and methylated reactions in a single tube.

36. The method of claim 34, wherein a QD donor-acceptor pair is QD525 and BODIPY, QD585 and Alexa594, or QD585 and Cy5.

37. The method of any of claims 1-20, wherein methylation is detected using a UV scanner.

38. A kit for MS-qFRET detection of DNA methylation, the kit comprising reagents for methylation-specific quantum dot fluorescence resonance energy transfer (MS-qFRET) selected from the group consisting of reagents for bisulfite conversion, reagents for PCR amplification, a first primer comprising biotin or another binding moiety, quantum dots (QDs) conjugated to a binding partner for the binding moiety; and instructions.

39. The kit of claim 38, further comprising a second primer labeled with a detectable moiety,

40. The kit of claim 38, wherein the instructions are for processing spectral information to determine the level of DNA methylation or diagnosing a disease characterized by an alteration in methylation.

41. A method for polynucleotide extraction and bisulfite conversion on a single reaction platform, the method comprising:
(a) contacting a sample on a reaction platform with a particle comprising a polynucleotide binding agent fixed to a magnetic or magnetizable element under conditions permissive for polynucleotide binding to said particle;
(b) isolating the polynucleotide:particle complex on said reaction platform;
(c) contacting the polynucleotide:particle complex with a bisulfite reagent under conditions permissive for the conversion of unmethylated cytosines to uracil in said reaction platform; and
(d) eluting the bisulfite treated polynucleotide from the particle within said reaction platform.

42. The method of claim 41, wherein the method further comprises detecting the methylation status of the polynucleotide in said reaction platform.

43. The method of claim 41, wherein the reaction platform is a reaction vessel or a reaction substrate.

44. The method of claim 41, wherein the reaction vessel is a tube, well, droplet, through-holes, micro or nanofluidic device.

45. The method of claim 41, wherein the reaction substrate is a membrane, filter, fiber, bead, gel matrix, chip, or glass slide.

46. A method for polynucleotide extraction and bisulfite conversion in a single reaction vessel, the method comprising:
(a) contacting a sample with a silica particle comprising a magnetic or magnetizable element under conditions permissive for polynucleotide binding to said silica particle in a reaction vessel;
(b) subjecting said silica particle to a magnetic field to isolate the polynucleotide:silica particle complex;
(c) contacting the polynucleotide:silica particle complex with a bisulfite under conditions permissive for the conversion of unmethylated cytosines to uracil; and
(d) eluting the bisulfite treated polynucleotide from the silica particle.

47. A method for polynucleotide extraction and bisulfite conversion in a single reaction vessel, the method comprising:
(a) contacting a sample with silica superparamagnetic particles (SSP) in a reaction vessel;
(b) isolating the SSP:DNA complex in said reaction vessel using a magnetic field;
(c) contacting the DNA with bisulfite in said reaction vessel under conditions permissive for the conversion of unmethylated cytosines to uracil;
(d) adjusting pH or salt conditions to induce formation of an SSP:DNA complex in said reaction vessel;
(e) isolating the SSP: bisulfite converted DNA complex in said reaction vessel using a magnetic field; and
(f) eluting the DNA from the SSP.

48. The method of any of claims 41-47, wherein the method further comprises detecting the methylation status of the polynucleotide in said reaction vessel.

49. The method of any of claims 41-47, wherein DNA methylation is detected using MS-qFRET or gel electrophoresis.

50. The method of any of claims 39-45, wherein the method increases DNA yield from 1000 to 7,000 percent relative to column based extraction.

51. The method of any of claims 41-47, wherein the method increases DNA yield from 3,500 to 7,000-percent relative to column based extraction.

52. The method of any of claims 41-47, wherein the method provides for detection of methylation in DNA extracted from about 10 μL whole blood.

53. The method of any of claims 41-47, wherein the method provides for detection of methylation in DNA extracted from about 200 μL of serum.

54. The method of any of claims 41-47, wherein the method yields about 40 to 70 ng/μL from about 200 μL of serum.

55. The method of any of claims 41-47, wherein the elution yield is about 70%, 75%, or 80% of the input DNA.

56. The method of any of claims 41-47, wherein the bisulfite conversion efficiency at four hours is about 20% or 25%.

57. The method of any of claims 41-47, wherein the sample is a biological sample or laboratory sample.

58. The method of any of claims 41-47, wherein the average recovery was at least about 70%, 75%, or 80%.

59. The method of any of claims 41-47, wherein the method requires about 4 hours.

60. The method of claim 47, wherein steps (a) and (d) are carried out at about pH 5-6.5 to permit SSP:DNA binding.

61. The method of claim 47, wherein step (f) is carried out at about pH 8-11.

62. A kit for methylation on beads, the kit comprising reagents selected from the group consisting of protease K, silica superparamagnetic particles (SSP), a washing buffer, and reagents for sodium bisulfite.

63. The kit of claim 60, further comprising directions for carrying out methylation on beads.
